(19) United States
(12) Patent Application Publication
(10) Pub. No.: US 2016/0145566 A1
(43) Pub. Date: May 26, 2016

(54) RECONFIGURABLE ASSEMBLY WITH FARADAY WAVE-BASED TEMPLATES

(71) Applicant: BRIGHAM AND WOMEN'S HOSPITAL, INC., Boston, MA (US)

(72) Inventors: Pu Chen, Cambridge, MA (US); Utkan Demirci, Stanford, CA (US)

(21) Appl. No.: 14/898,297

(22) PCT Filed: Jun. 20, 2014
(86) PCT No.: PCT/US14/43319

§ 371 Date: Dec. 14, 2015

Related U.S. Application Data
(60) Provisional application No. 61/837,687, filed on Jun. 21, 2013.
(60) Provisional application No. 61/888,812, filed on Oct. 9, 2013.

(51) Int. Cl. C12N 5/00; C12M 3/00; C12M 1/42; C12N 13/00

(52) U.S. Cl. CPC C12N 5/0062; C12N 13/00; C12M 21/08; C12M 35/04

(57) ABSTRACT

A method manufactures a structure based on reconfigurable assembly with faraday wave-based templates. The method includes the steps of providing a chamber containing a gas-liquid interface or liquid-liquid interface and dispersing a plurality of floaters at the gas-liquid interface or liquid-liquid interface. The method further includes oscillating the chamber along an axis orthogonal to the gas-liquid interface or liquid-liquid interface, thereby generating a standing wave formed by a parametric instability on the surface of the liquid. After formation of the standing wave, the floaters are allowed to self-assemble, at which point the floaters can be linked together.

### CROSS REFERENCE TO RELATED APPLICATIONS

This application is based on, claims the benefit of, and incorporates herein by reference U.S. Provisional Application No. 61/837,687, filed Jun. 21, 2013 and U.S. Provisional Application No. 61/888,812, filed Oct. 9, 2013.

### STATEMENT REGARDING FEDERALLY SPONSORED RESEARCH

This invention was made with government support under 1150733 awarded by the National Science Foundation. The government has certain rights in the invention.

### BACKGROUND OF THE INVENTION

Nanoscale self-assembly has been widely explored in recent decades, and it has had a significant impact on various fields, such as supramolecular chemistry, crystallization and nanofabrication. In contrast, micrometer-scale self-assembly is an emerging field with great promise for applications, such as fabrication of artificial tissues in a bottom-up manner, development of smart modular microrobots and assembly of microelectronic circuits. A limited number of mechanisms have been reported for micrometer-scale self-assembly, such as magnetic attraction, capillary force and electrostatic interactions. However, there are no described mechanisms that enable parametrically-reconfigurable generation of diverse structures in a scalable and parallel manner. This presents a major challenge for developing practical applications for micrometer-scale self-assembly.

Self-assembly of micrometer-scale building blocks into structurally ordered and functionally diverse systems has the potential to impact methodologies in a broad variety of fields, such as modular microrobots, microelectronics, and tissue engineering.

Tissue engineering, in particular, holds great promise to provide regenerative therapies and research platforms for clinical applications. Most tissues in human body are composed of repeating basic cellular structures (i.e., tissue functional units), such as the hepatic acinus in the liver, the nephron in the kidneys, and islets in the pancreas. The ability to generate three-dimensional (3D) tissue functional units is of benefit for diverse tissue engineering applications in therapeutics, diagnostics and drug screening. However, despite significant promise, a number of challenges constrain the generation of functionalized 3D tissue units for practical applications. These include: (i) the inability to create repeating complex 3D zonal architectures; (ii) the organization of the cells and their surrounding microenvironments with microscale resolution in the engineered tissue functional units; and (iii) the difficulty in obtaining sufficient vascularization within the graft to minimize necrosis and loss of function. Therefore, there is a need for self-assembly systems and methods for the manipulation and templating of micrometer-scale building blocks.

### SUMMARY OF THE INVENTION

The present invention overcomes the aforementioned drawbacks by providing system and method by which micrometer-scale objects at the gas-liquid interface self-assemble into diverse sets of ordered, symmetric structures with high repeatability and stability by Faraday waves, which are used as a scalable, reconfigurable and globally shape-controllable template. This process is referred to herein as Reconfigurable Assembly with Faraday wave-based Templates (RAFT). The RAFT system exhibits unique dynamic self-assembly properties, including self-healing, self-adaptation and directional selectivity. The study of these properties is fundamentally important to enhance our understanding of the collective behavior of floaters in emergent structures or complex systems in nature.

RAFT is an advantageous fabrication method with real-world applications. Compared with conventional molding techniques where one solid mold is fabricated for one part, the mechanisms described herein enable parametrical reconfiguration of the liquid templates in a parallel format. Moreover, it is anticipated that the described RAFT mechanisms can be used to generate any predefined complex structures by multiple-frequency forcing Faraday waves.

In another embodiment, a system and method are provided to generate vascularized 3D tissue functional units by merging microscale self-assembly technologies, lab-on-a-chip (LOC) technologies, hydrogel engineering technologies and tissue engineering principles. The system and methods can be implemented to engineer a broadly applicable high-throughput microscale assembly platform for the generation of functional tissue constructs in vitro. In one aspect, tissue constructs are created that are composed of repeating functional units, with microscale spatial control over the matrix, encapsulated cells, and growth factor distribution. In another aspect, the system and methods include the aforementioned RAFT system. The topography of the gas-liquid interface can be used as a template, which can be parametrically controlled by frequencies and amplitudes of Faraday waves. Microscale hydrogel units can specifically be assembled on a liquid template into a monolayer structure, which can be further stacked layer-by-layer into a 3D architecture. By assembling cell-encapsulating hydrogel units into 3D constructs and culturing them for further maturation, hydrogel scaffolds can degrade and be completely replaced by cellular growth and extracellular matrix (ECM) deposition, resulting in formation of 3D native like tissue constructs. The LOC technologies can be developed to interface with the engineered tissue functional units and provide a simulated microphysiological environment for further tissue functionalization. In vivo evaluation of an engineered graft can be performed with ectopic implantation in a mouse model.

In one embodiment, a method is provided for manufacturing a structure. The method includes the steps of (i) providing a chamber containing a gas-liquid interface or liquid-liquid interface; (ii) dispersing a plurality of floaters at the gas-liquid interface or liquid-liquid interface; (iii) oscillating the chamber along an axis orthogonal to the gas-liquid interface or liquid-liquid interface, thereby generating a standing wave at the gas-liquid interface or liquid-liquid interface; (iv) allowing the floaters to self-assemble; and (v) linking the floaters, wherein the standing wave is formed by a parametric instability on the surface of the liquid.

In one aspect, the standing wave is a Faraday wave. In another aspect, the floaters have a diameter of about 0.1 μm to about 1 m. In yet another aspect, floaters have a diameter of about 10 μm to about 5 mm. In still another aspect, the floaters are biological samples (including microorganisms, cells, cell clusters, cell spheroids, cell fragments, viruses, bacteria, fungi, peptides, nucleic acids, proteins, carbohydrates, secreted cellular products and exosomes), chemical samples (including biomaterial units such as hydrogel units and polymer units), and non-biomaterial units (such as semiconductor units and metallic units). In another aspect, the step of linking the plurality of floaters further comprises photo cross-linking, ultra-violet (UV) cross-linking, chemical cross-linking, thermo cross-linking, surface molecule recognition-based linking, or geometric shape-based linking.

In a further aspect, the method further includes the steps of forming a monolayer structure following the step of linking the plurality of floaters; repeating the method of claim 8 to produce a plurality of monolayer structures; and stacking the monolayer structures layer by layer into a 3D architecture. In another aspect, the method further includes the step of culturing the 3D architecture, thereby forming 3D tissue constructs. In still another aspect, the method includes forming a monolayer structure following the step of linking the plurality of floaters. In another aspect, a structure can be made by the method.

In a second embodiment, a system is provided for manufacturing a structure, including a chamber having a bottom surface; a liquid disposed in the chamber; a plurality of floaters disposed on the liquid; an oscillating mechanism configured to oscillate the chamber along an axis orthogonal to the gas-liquid interface or liquid-liquid interface, thereby generating a standing wave at the gas-liquid interface or liquid-liquid interface; and a linking mechanism configured to link the plurality of floaters. The standing wave is formed by a parametric instability on the surface of the liquid.

In one aspect, the standing wave is a Faraday wave. In another aspect, the plurality of floaters has a diameter of about 10 μm to about 5 mm. In yet another aspect, the floaters are biological samples (including microorganisms, cells, cell clusters, cell spheroids, cell fragments, viruses, bacteria, fungi, peptides, nucleic acids, proteins, carbohydrates, secreted cellular products and exosomes), chemical samples (including biomaterial units such as hydrogel units and polymer units) and non-biomaterial units (such as semiconductor units and metallic units). In still another aspect, at least a portion of the floaters encapsulate at least one biological sample. In another aspect, at least one biological sample is a microorganism, a cell, a cell cluster, a cell spheroid, a cell fragment, a virus, a bacterium, a fungus, a peptide, a nucleic acid, a protein, a carbohydrate, a secreted cellular product or an exosome.

In another aspect, the system further includes a substrate for stacking a plurality of monolayer structures layer by layer into a 3D architecture. In still another aspect, the system includes a culture chamber for culturing the 3D architecture into 3D tissue constructs.

In a third embodiment, a method is provided for manufacturing a structure. The method includes the steps of (i) providing a chamber containing a gas-liquid interface or liquid-liquid interface; (ii) dispersing a plurality of floaters at the gas-liquid interface or liquid-liquid interface, the floaters having a diameter of about 10 μm to about 5 mm; (iii) oscillating the chamber along an axis orthogonal to the gas-liquid interface or liquid-liquid interface, thereby generating a Faraday wave at the gas-liquid interface or liquid-liquid interface, the Faraday wave formed by a parametric instability on the surface of the liquid; (iv) allowing the floaters to self-assemble; and (v) linking the floaters to form the structure.

The acoustic system can be used also for sorting cells based on their response to the waves and based on characteristics such as density and geometry.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 shows a comparison of macroscopic structures of generated patterns in term of complexity and diversity including patterns generated by Faraday-wave-templated self-assembly mechanism. All floaters are 200 μm in diameter.

FIG. 2 shows templated self-assembly by Faraday waves in shape-varied chambers. Black dashed lines indicate the outline of the chamber. All scale bars on the lower right hand side of the self-assemblies represent 4 mm.

FIGS. 3A-3Q show a demonstration of Faraday wave forms. FIGS. 3A-3D are schematic drawings of standing wave lattices under different wave forms. The lines (grids in FIGS. 3A, 3C and 3D, and thin vertical lines in 3B) indicate the nodes of standing wave lattices, and the spots indicate phase reversed antinodes. The corresponding assembled structures are individually indicated by a dashed square with a label in the schematic drawings. FIGS. 3E-3G and 3N show assembled structures generated using the square form of the Faraday waves. FIGS. 3I-3L show assembled structures generated by stripe form of Faraday waves. FIGS. 3M-3Q show assembled structures generated with two other crystal-like forms of the Faraday waves. SQ: square form of the Faraday waves; ST: stripe form of the Faraday waves; CR1 and CR2: crystal-like forms of the Faraday waves. All scale bars represent 2 mm.

FIG. 4A-4C show spatial phase and order of assembled patterns. FIG. 4A shows assembly of polystyrene beads into different spatial phases under the 2nd, 3rd and 4th order in the first, second, and third columns, respectively. All scale bars represent 2 mm. FIG. 4B shows the relationship between the stable standing-wave modes for the square form of Faraday waves. The simulation results were obtained using Eq. 2, below. The color bar depicts the amplitude of the standing waves. The code [φ/45°, φx/90°, φy/90°] represents the standing-wave mode. λis the Faraday wavelength, and (λ/4, λ/4) indicates translation of the standing wave lattice by λ/4 in both x-axis and y-axis directions. The lines of symmetry are indicated by white dash-dotted lines. FIG. 4C shows merged images of the bead assembled patterns and the corresponding simulated standing wave lattices. Dashed lines in row [1,0,0] highlight the similarity of the assembled structures within the same standing wave mode.

FIGS. 5A-5E show a principle demonstration of Faraday wave-directed self-assembly. FIG. 5A shows the system before the self-assembly. FIG. 5B shows wave-floater interactions. FIG. 5C shows global pattern formation of the self-assembly. FIG. 5D shows floater-floater interactions. FIG. 5E shows formation of the closely packed self-assembled structure; enlarged local zone features bead arrangements within the assembled structure.

FIG. 6 shows a classification method for Faraday wave-directed assembly patterns.

FIGS. 7A-7C show scalability of templated self-assembly by Faraday waves. Self-assembly of polystyrene beads (200 μm) into the structure SQ[0,0,0:4]. FIG. 7A shows a 10 mm×10 mm chamber, FIG. 7B shows a 20 mm×20 mm chamber, and FIG. 7C shows a 30 mm×30 mm chamber. All scale bars represent 2 mm.

FIG. 8 shows the relationship between the Faraday wave frequency and the characteristic length of the assembled structures. The characteristic lengths are given as the means±standard deviation (n=4); Faraday wave frequency is given as a range for the corresponding assembled structures.

FIG. 9 shows a phase diagram for the assembly pattern formation. Each code between the dashed lines indicates a stable assembled structure. The code SQ[mode:order] represents assembled structures using the square form of Faraday wave. Other codes are for structures generated by stripe (ST) and other crystal-like (CR) forms of the Faraday waves. The data are presented as means±standard deviation (n=3). The dimension of the carrier solution chamber is 10 mm (length)×10 mm (width)×1.5 mm (height).

FIG. 10 shows a phase diagram of assembled structures as a function of the Faraday wave frequency and vibrational acceleration. The dimensions of the carrier-solution chamber is 20 mm (length)×20 mm (width)×1.5 mm (height). “*” indicates rotational symmetry; otherwise, reflection symmetry is presented. The data are presented as means±standard deviation (n=3).

FIGS. 11A-11D shows the reconfigurability of Faraday wave-directed self-assembly. FIG. 11A shows a demonstration of the principle of dynamic self-assembly by Faraday waves: (f_{A}, a_{A}) and (f_{B}, a_{B}) are vibrational frequencies and accelerations for the formation of structures A and B, respectively. FIGS. 11B-11C show a dynamic process of pattern formation and transformation controlled through adjustment of the input vibrational parameters. Top-down view (FIG. 11B), and side view (FIG. 11C). I-VI show different stages of self-assembly: I. before assembly; II. during assembly; III. formation of the ring-shaped structure; IV. intermediate state; V. formation of “H”-shaped structure; VI. restoration of the ring-shaped structure. FIG. 11D, Time evolution of the assembly fraction during the self-assembly process. Vibration was applied at time zero. The assembly fraction is calculated as the ratio of the number of assembled beads to the number of total beads. “*” indicates resetting vibrational parameters: the vibrational frequency was first increased from 46 Hz to 60 Hz, and the acceleration was then increased from 1.46 g to 1.9 g. “**” indicates resetting vibrational parameters: the acceleration was first decreased from 1.9 g to 1.46 g, and the vibrational frequency was then decreased from 60 Hz to 46 Hz. All of the experiments were performed in a 10 mm×10 mm×1.5 mm chamber using 200 μm beads. All scale bars represent 2 mm. Note: Top views and side views were recorded separately and approximately correspond to each other in the corresponding stages.

FIG. 12 is a schematic demonstration of latency time and assembly time.

FIGS. 13A-13I show a characterization of time evolution of the macroscopic and microscopic structures during self-assembly. FIGS. 13A-13C show the effect of vibrational acceleration, vibrational frequency and bead coverage on the time evolution of the macroscopic structure during self-assembly. FIGS. 13D-13E show one-way ANOVA analysis of vibrational acceleration and frequency effects on the self assembly. The data are presented as means±standard deviations (n=3). *P<0.05. FIGS. 13F-13I show the effect of vibrational acceleration, vibrational frequency and bead coverage on the time evolution of bead arrangements during self-assembly. All the curves are presented as mean±standard deviation (n=3).

FIG. 14 shows a comparison of microscopic structures of generated patterns in terms of particle arrangements. Dashed rectangles are enlarged at the right bottom. Particle size is 200 μm for the left figure and 100 μm for the right figure. Scale bars represent 2.5 mm.

FIG. 15 shows a demonstration of neighbor number (CO and corresponding bead arrangements.

FIGS. 16A-16L show a generalization and applicability of Faraday wave directed self-assembly. FIGS. 16A-16C show self-assembly of microscale GelMA hydrogel units into node patterns. FIGS. 16D-16F show self-assembly of PDMS blocks into node patterns. FIGS. 16G-16I show self-assembly of beads and copper powders into complementary patterns. FIGS. 16J-16L show self-assembly of silicon chiplets into antinode patterns. All of the experiments were performed in a 20 mm×20 mm×1.5 mm chamber.

FIG. 17 shows two-frequency forcing Faraday waves for templated self-assembly.

FIG. 18 shows templated self-assembly by Faraday waves. On the left, 100 mm×100 mm×0.5 mm (Aspect ratio, Γ=200) with 500 μm beads. On the right, 20 mm×20 mm×1.5 mm (Aspect ratio, Γ=13.3) with 25 μm beads. Scale bar represents 4 mm.

FIG. 19 shows floater size effect on self-assembly. Hexagonal-shaped PEG hydrogels with a size 0.5, 1, 2, and 5 mm in diameter from left to right. Scale bar represents 4 mm.

FIG. 20 shows an experimental demonstration of parallel manufacturing in 2-by-2 chamber arrays. Scale bar represents 10 mm.

FIGS. 21A-21B show an experimental setup. FIG. 21A shows experimental setup for digital SLR camera. The top left insert shows the length of the carrier solution chamber, L is 10 mm and 20 mm. The top right insert shows the device assembly. FIG. 21B shows an experimental setup for the high-speed camera.

FIG. 22 shows a procedure for fabrication of PDMS building blocks.

FIGS. 23A-23E show control groups for phase diagrams. Assembly onset acceleration of “H”-shaped structures (SQ[0,0,1:2]) at 60 Hz was used as a reference for all of the control experiments. The experiments were conducted in a 10 mm by 10 mm carrier solution chamber. FIG. 23A shows the effect of initial acceleration on the assembly onset acceleration; FIG. 23B shows the effect of acceleration ramp rate on the assembly onset acceleration; FIG. 23C shows the effect of solution density on the assembly onset acceleration; FIG. 23D shows the effect of solution thickness on the assembly onset acceleration; FIG. 23E show the effect of bead coverage on the assembly onset acceleration. The data are plotted as the mean±standard deviation (n=3). One-way ANOVA analysis with Turkey test: “**” bracketing indicates no significance (P<0.05).

FIG. 24 shows a principle demonstration of Faraday wave directed self-assembly for tissue engineering.

FIG. 25 shows an experimental demonstration of Faraday wave directed self-assembly for tissue engineering.

FIGS. 26A-26C show the effect of Faraday waves on cell viability. FIG. 26A shows fluorescence images of cells for one and three days' cell culture. Bright spots indicate primarily live cells. FIG. 26B shows a quantitative analysis of cell viability after one day and three days of cell culture. FIG. 26C shows the effect of exposure time and acceleration on cell viability. Cells were cultured 24 hours before live/dead assays. n=4. Error bars represent ± standard deviation; p<0.05.; “N.S.” bracketing indicates the difference is not significant.

FIG. 27 shows a principle demonstration for fabrication of microscale hydrogel units.

FIG. 28 shows a conceptual demonstration of Faraday wave-based templated self-assembly technology for engineering of 3D tissue functional units.

FIG. 29 shows a schematic diagram of hepatic acinus.

FIG. 30 shows periodic structures of Faraday waves and more specifically, single-scale Faraday waves (reflection photos).

FIGS. 31A-31D show self-assembly technologies. FIG. 31A shows magnetic assembly of hydrogel units into different shapes. FIG. 31B shows magnetic assembly of hydrogel units layer by layer. FIG. 31C shows Faraday wave assembly of polystyrene (PS) beads, NIH 3T3 cells, and PEG hydrogel units (from left to right: 50 μm PS beads; 25 μm PS beads; 10 μm NIH 3T3 cells; 300 μm PEG hydrogel units). FIG. 31D shows specific assembly of floaters based on wettability (leftmost image: hydrophilic polystyrene beads and hydrophobic copper powders) and geometry (third image from left: circled, ratio=3; uncircled, ratio=0.3). The second and fourth pictures from the left are numerical simulations of corresponding standing waves. Intensity bar indicates wave amplitude. Scale bars represent 4 mm. (Tasoglu, S. et al., Adv. Mater., 25 (8), 1081, 2013)

FIGS. 32A-32H show hydrogel engineering technologies. FIGS. 32A-32C show engineered hydrogel units with different sizes (FIG. 32A), shapes (FIG. 32B) and magnetic properties (FIG. 32C). FIG. 32D shows engineered hydrogel units with different types of cells. FIG. 32E shows results for cell viability assays. FIG. 32F shows immunostaining of cells in hydrogel units. FIG. 32G shows a quantitative plot of the cell type ratio in hydrogel units. FIG. 32H shows cell proliferation assays. (Gurkan, U. A., et al., Adv. Mater., 25 (8), 1192-1198, 2013; Tasoglu S., et al., Adv. Mater., 25 (8), 1137-1143, 2013; Xu, F., et al., Biomaterials, 32 (31), 7847-7855, 2011)

FIG. 33 shows procedures for engineering periodic hydrogel structure. Step a, simplify native tissue into a standard histological model. Step b, convert the histological model into a designed pattern for assembly. Step c, decompose the designed pattern into a sum of sine waves. Step d, generation of liquid-based template based on the frequencies of the sine waves. Step e, assembly of microscale hydrogel units using the liquid-based template.

FIGS. 34A-34D show optical images of cell assembly by RAFT. FIGS. 34A and 34B is an image of self-assembled cell-encapsulated hydrogel structures in a 20 mm² chamber. FIG. 34C is an image of cell-encapsulated hydrogel structures in a 20 mm² chamber without assembly (no assembly control). In each of FIGS. 34A-34C, cells were stained with methylene blue for visualization and the scale bars represent 2 mm. FIG. 34D shows a microscopic fluorescence image of self-assembled cell-encapsulated hydrogel structures. Assembled structures were cross-linked by UV exposure. Cells were stained with carboxyfluorescein diacetate, succinimidyl ester (CFSE). The scale bar represents 200 μm.

FIGS. 35A-35J show optical images of cells adhered to microcarrier beads following self-assembly and chemical cross-linking. FIGS. 35A is a bright field image of the overall structure achieved with RAFT. FIG. 35B is a fluorescence image of the overall structure achieved with RAFT. For FIGS. 35A and 35B, cells were stained with CFSE and images were recorded after one day of tissue culture. FIGS. 35C-35F show images of live dead staining indicating greater that 90% cell viability after five days of tissue culture. FIGS. 35C and 35E are fluorescence field images, and FIGS. 35D and 35F are bright field images. FIGS. 35G-35J show images acquired with a confocal microscope after five days of tissue culture. FIG. 35H highlights cell nuclei detected with 4′,6-diamidino-2-phenylindole (DAPI) staining. FIG. 35H highlights live cells detected with calcein-AM. FIG. 35I highlights dead cells detected with ethidium homodimer-1. FIG. 35J is a composite image of FIGS. 35G-35I showing cell spreading on the beads.

FIGS. 36A-36L show data collected for tissue engineering experiments incorporating RAFT techniques. FIGS. 36A-36D show assembly of cell-seeded microcarrier beads. FIG. 36A is a fluorescence image of microcarrier beads with CFSE stained NIH 3T3 fibroblast cells after assembly and cross-linking. FIG. 36B is a fluorescence image of a live/dead assay of the cells of FIG. 36A seeded on the microcarrier beads following 3 days of culture after chemical cross-linking. Calcein-AM was used to detect live cells, while ethidium homodimer-1 was used to detect dead cells. FIGS. 36C and 36D show formation of 3D neural structures on assembled microcarrier beads after 14 days of cell culture. FIGS. 36E and 36H show scaffold-free assembly of cells spheroids (mean size: 200 μm). FIG. 36F is an image of a magnified region of FIG. 36E as indicated with dashed lines. FIGS. 36G and 36H show bright field images of assembled structures from cell spheroids. FIG. 36I-36L show scaffold-free assembly of fibroblast cells and cytocompatibility tests. FIG. 36I is a fluorescence image of cells stained with CFSE. FIG. 36J is an image of a magnified region of FIG. 36I as indicated with dashed lines. FIG. 36K is a bar graph illustrating cell viability data under assembly onset acceleration at various vibrational frequencies (n=6). FIG. 36L is a plot showing cell number (as determined with Alamar Blue) as a function of time for cells exposed to 15-second agitations at 50, 100 and 200 Hz. Exposed cells were seeded in a 64-well plate with a seeding density of 200 cells/well for 11 days of cell culture. Data is presented as mean±S.D (n=8).

FIGS. 37A-37D show formation of 3D neural structures by assembling and stabilizing neuron-seeded microcarrier beads with RAFT. FIG. 37A is a fluorescence image of immunostaining with DAPI, FIG. 37B is a fluorescence image of immunostaining with Nestin, FIG. 37C is a fluorescence image of immunostaining with Neun, and FIG. 37D is a merged image of FIGS. 37A-37C. The scale bar in FIG. 37A represents 200 μm.

FIGS. 38A-38D show formation of 3D neural structures by assembling and stabilizing neuron-seeded microcarrier beads with RAFT. FIG. 38A is a fluorescence image of immunostaining with DAPI, FIG. 38B is a fluorescence image of immunostaining with MAP2, FIG. 38C is a fluorescence image of immunostaining with Neun, and FIG. 38D is a merged image of FIGS. 38A-38C. The scale bar in FIG. 38A represents 200 μm.

FIGS. 39A-39H show bright filed images of the application of RAFT to cell spheroids formed into various patterns. Assembly was performed in OptiPrep-PBS solution (density: 1.1 g mL⁻¹) in a square chamber (20 mm×20 mm×1.5 mm). Cell spheroids were about 200 μm in diameter.

FIGS. 40A-40D show fluorescence images of RAFT applied to cells stained CellTrace™ CFSE. FIG. 40A shows cells uniformly dispersed on a surface (control group). FIG. 40B-40D show the application of RAFT to cells in OptiPrep™-PBS solution (density, 1.2 g mL-1). Scale bars represent 1 mm.

### DETAILED DESCRIPTION OF THE INVENTION

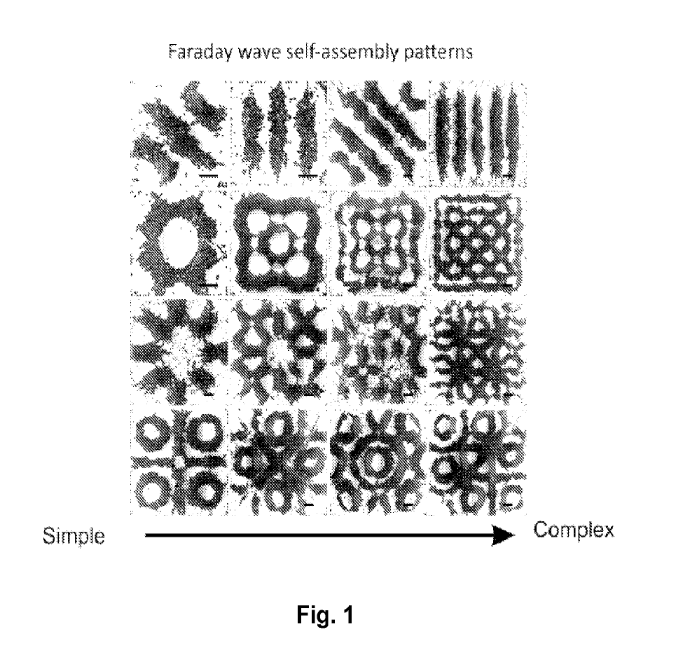

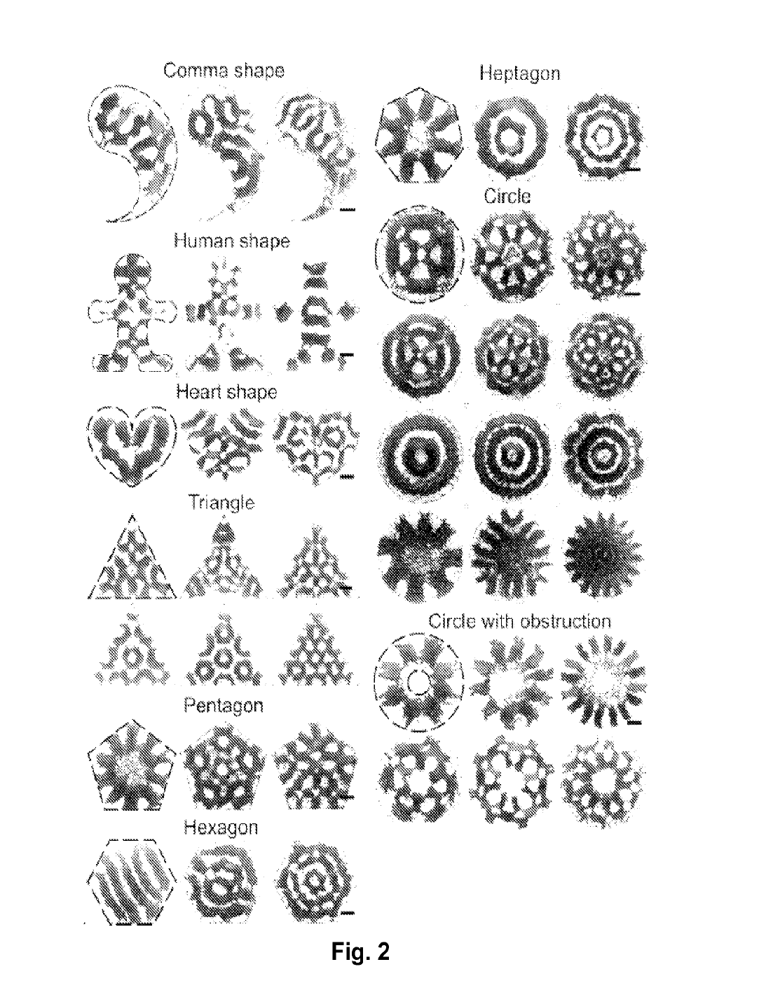

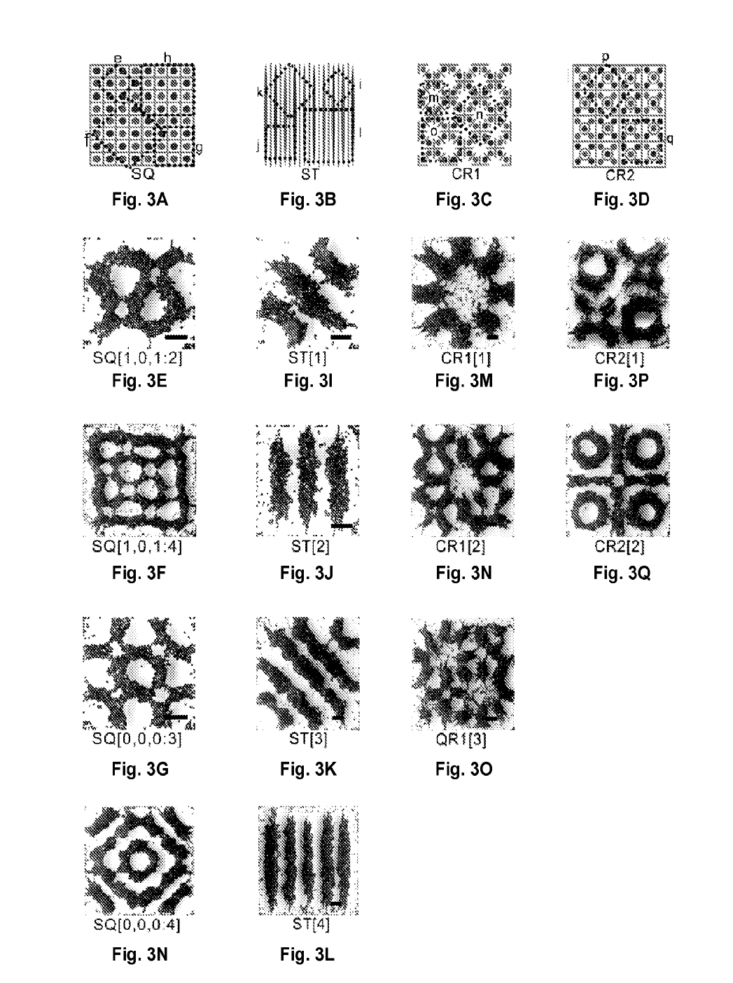

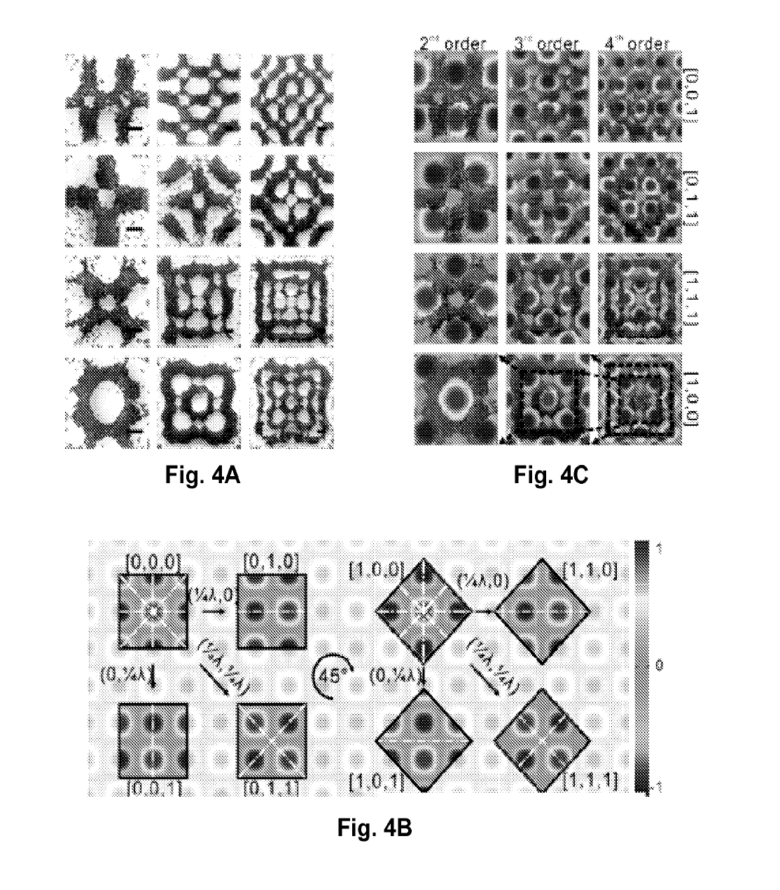

The demonstrated RAFT mechanisms can generate diverse macroscopic structures (FIG. 1) from simple to complex by parametrically configuring the topography of standing waves in the chambers. The diversity of the assembled structures originates from combinations of: (i) symmetry of the chamber (FIG. 2); (ii) the wave form (FIG. 3); (iii) the spatial phase of Faraday waves in the chamber (FIG. 4C); and (iv) the harmonic order of the Faraday waves in the chamber (FIGS. 4A, 4B).

In one embodiment, a RAFT system consists of micrometer-scale floaters suspended at the gas-liquid interface of a carrier solution in an open square chamber, which is vertically vibrated in a sinusoidal manner by a vibration generator. Faraday waves (i.e., standing waves at the gas-liquid interface) are excited by this vertical vibration. By controlling the vibrational parameters (i.e., the frequency and the acceleration from the vibration generator), diverse sets of geometric shaped Faraday waves (e.g. stripes, squares) can be obtained in the chamber. Examples of floaters can include any type of particle having a diameter of about 0.1 μm to about 1 m. Floaters can be biological materials such as cells and cell components, or other materials such as polystyrene beads and hydrogel blocks. Other types of floaters can also be used as will be detailed herein.

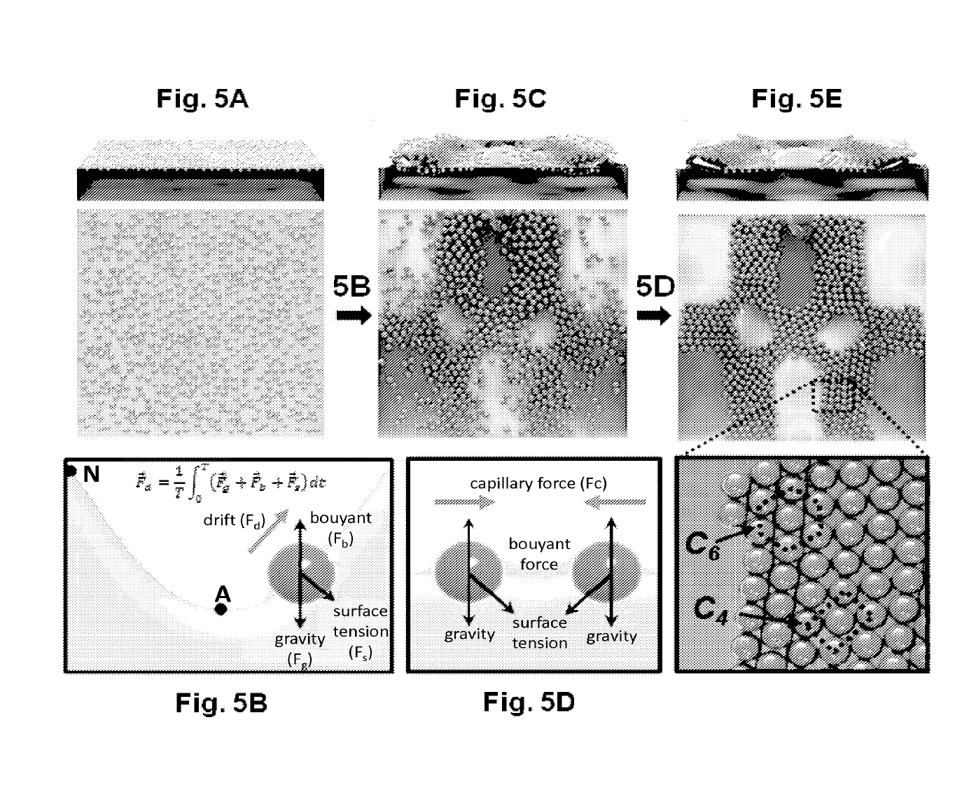

Faraday-wave-directed self-assembly of floaters in the RAFT system is enabled by both standing wave-floater and floater-floater interactions (FIG. 5). Standing waves arise from parametric instability due to vertical vibration and arrange in a lattice format in the chamber due to side-wall boundary effects (boundary constrained lattice) or fluid properties (boundary unconstrained lattice). The floaters suspended on the standing waves drift and concentrate to either the nodes (amplitude minima) or antinodes (amplitude maxima) of the standing waves, depending on the direction of the drift force (i.e., the vector sum of the gravitational force, the buoyant force and the surface tension force averaged during one wave period). For floaters in an embodiment of the RAFT system, the drift force points to either nodes or antinodes of the standing waves based on their relative density and wettability to carrier solution (FIG. 5B), which results in a global pattern formation of a bead assembly that covers the nodal areas (FIG. 5C). In addition, the attractive capillary interactions among the beads closely pack the assembled global structures (FIG. 5D), which results in the formation of ordered microscopic structures (e.g., hexagonal/square close packing) (FIG. 5E). These capillary interactions also maintain the assembled structure by the hydrodynamic-flow-induced Stokes drag force that tends to break up assembled structures.

In one aspect, The drift energy, U, experienced by a floater with a radius R in an applied standing wave can be simply described by Eq. 1 as follows, assuming no boundary effects on the standing waves,

$$U = [ \frac{4}{3} π {R}^{3} {ρ}_{par} - π {ρ}_{liq} R {δ}^{2} + π {ρ}_{liq} \frac{{δ}^{3}}{3} ] \frac{{ω}^{2}}{4} {\vert ζ \vert}^{2} + \frac{3 {π}^{2} μδ α}{ω} \vert {ζ}_{90 °} \vert \qquad ( Eq . 1 )$$

where R is the floater radius, ρₚₐᵣ is the floater density, ρ_{liq} is the liquid density, δ is the submerged length of the floater in the liquid which is a function of R, ρₚₐᵣ, ρ_{liq} and the contact angle of the floater θ, ω is the angular frequency of the standing waves, ζ is the deformation of the fluid surface, μ is the dynamic viscosity of the liquid, α is the driving acceleration, and ζ_{90°} is the deformation with 90° phase shift in x and y directions. Briefly, distribution of the drift energy is sensitive to contact angle and density ratio of the floater to fluid and is not sensitive to floater size and shape. Small contact angle and low density normally result in regions with the lowest drift energy on nodes of standing waves, while large contact angle and high density result in regions with the lowest drift energy on antinodes of standing waves. Lateral capillary forces also take effect on the local arrangement of floaters when floaters are near each other. These capillary forces don't contribute to formation of the resulting global structure and thus are not included in the theoretical model.

Particularly, for polystyrene divinylbenzene beads in the standing waves, the lowest drift energy exists on nodal region, resulting in nodal patterns with different coverage rates. For copper-zinc powder in the same standing waves, the lowest drift energy exists on antinode region, resulting in antinode patterns. In one aspect, by introducing two types of microscale materials with complementary distributions of drift energy, complementary patterns of two types of materials may be achieved.

RAFT experiments were performed in a chamber with varied reflection symmetries. Chambers have a surface area of approximately 400 mm² and a depth of 1.5 mm. Stable assembled patterns were achieved in all chambers by varying the frequency band from 30 Hz to 70 Hz (FIG. 2). It was observed that most assembled patterns have a symmetry equal or less than the symmetry of the corresponding chamber with few exceptions (e.g., heptagon). Additionally, it was observed stable, assembled patterns were more readily achieved in square-, circle- and triangle-shaped chambers than in chambers with other geometries. Self-assembly was also achieved in chambers with obstructions (FIG. 2).

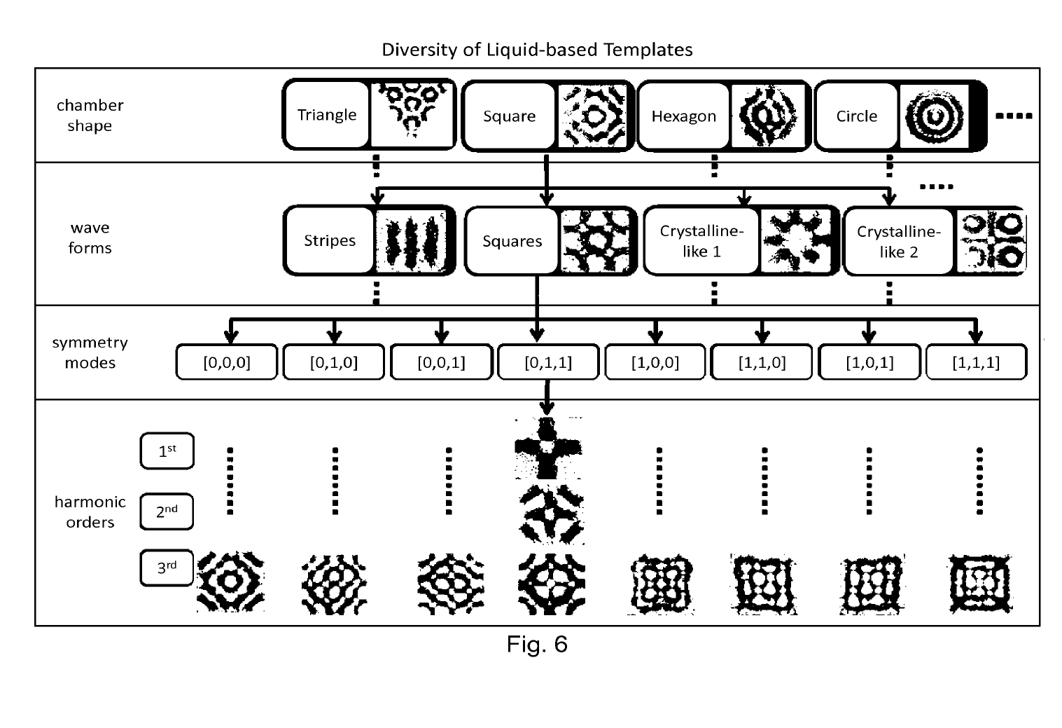

A remarkable diversity of assembled structures was achieved by changing the vibrational parameters (FIGS. 3, 4A). The diversity of the assembled global structures originates from combinations of the wave form, the symmetry mode and the harmonic order of the Faraday waves in the carrier-solution chamber (FIG. 6). Faraday waves can take on the waveforms of squares, stripes and other two-dimensional crystal-like structures (FIGS. 3A-3D). For individual forms of Faraday waves, there are several stable standing wave modes that are determined by the symmetry and direction of the standing wave lattice in the chamber. The stable modes for all waveforms preserved at least one-fold reflection or two-fold rotational symmetry. In addition, each mode includes several standing wave lattices with different harmonic orders, which are defined as the number of half Faraday wavelengths in the chamber. By analyzing the square form of the Faraday waves as a model, eight types of standing wave modes were identified with one-, two- or four-fold reflection symmetries (FIG. 4). The relationship between these modes can be understood by translating the standing wave lattice by one-quarter wavelength in the x-axis and/or the y-axis direction or by rotating the standing waves about the center of the chamber by 45° (FIG. 4B). The topography of a stable standing wave lattice in the square chamber can be described by Eq. 2 as follows, assuming no physical boundary:

$$ζ ( x , y ) = A cos [ \frac{m π ( x cos ϕ - y sin ϕ )}{L} + {ϕ}_{x} ] \times cos [ \frac{n π ( y cos ϕ + x sin ϕ )}{L} + {ϕ}_{y} ] \qquad ( Eq . 2 )$$

where ‘ξ(x,y)’ is the local wave elevation; ‘A’ is the wave amplitude; ‘L’ is side length of the square chamber; ‘x’ and ‘y’ are the coordinates of the points; ‘m’ and ‘n’ are the numbers of the half wavelengths in the x and y directions, which correspond to the length and width, respectively (m is equal to n in the square form of the Faraday waves); ‘φₓ’ and ‘φ_{y}’ are the phase angles in the x and y directions, respectively; and ‘φ’ is the rotation angle about the center of the chamber. For stable standing wave lattices, φₓ and φ_{y} can be 0° or 90°, and φ can be 0° or 45°. For example, a few of the assembled structures generated the square form (FIG. 4A) and their corresponding images merged with the simulated results (FIG. 4C) are provided based on symmetry modes (rows) and harmonic orders (columns). These results show close agreement between the experiments and the simulations. Other assembled structures and the corresponding schematic diagram of the standing wave lattice are depicted in FIG. 3. Taken together, these results indicate that highly diverse RAFT patterns can be generated by manipulating the geometry of the chamber.

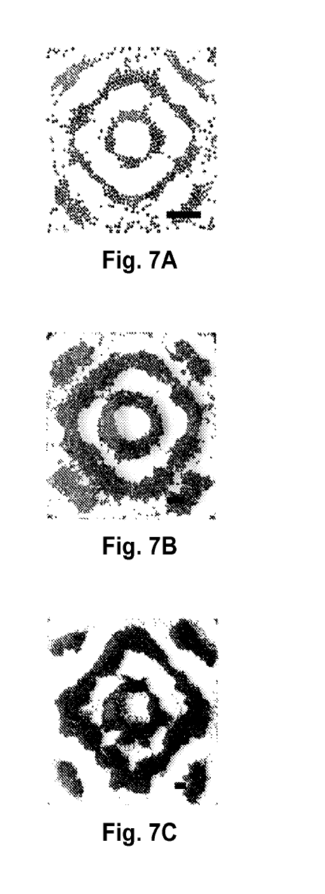

For each waveform type, the assembled structures exhibit surprising similarities in their geometries. First, simple patterns periodically repeat themselves within a single complex assembled structure (e.g. the cross shape in FIG. 4A: first row at second column, and the ring shape in FIG. 3Q). Second, the assembled structures generated by low-order standing wave lattices (e.g., second order) were observed to be a repeating portion of the assembled structures generated by higher-order standing wave lattices (e.g. third and fourth order) under the same symmetry mode (see dashed squares in FIG. 4C). Third, an assembled structure is scalable in dimension when the same standing waves lattice is generated in square chambers with different sizes (e.g., 10 mm and 20 mm) (FIG. 7). All these similarities between the assembled structures can be understood based on the corresponding standing wave lattices (FIG. 4C). These combined observations are indicative that the RAFT system, in one embodiment, enables scalable, parallel material manufacturing with complex patterns.

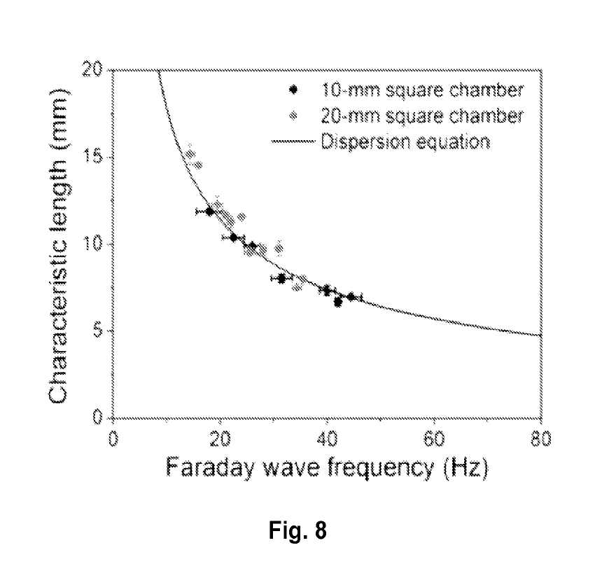

To further quantitatively analyze the geometry of these assembled structures under the square wave form, a characteristic length was defined as the length of the repeating units in the assembled structures. It was found that the relationship between a characteristic length and the corresponding Faraday wave frequency (half of the vibrational frequency) closely follows the inviscid dispersion equation (Eq. 3) that describes the relationship between the Faraday wavelength and the Faraday wave frequency (FIG. 8):

$${( λ f )}^{2} = [ \frac{g λ}{2 π} + \frac{σ}{{ρ}_{l}} ( \frac{2 π}{λ} ) ] tanh ( \frac{2 π H}{λ} ) \qquad ( Eq . 3 )$$

where ‘f’ is the Faraday wave frequency; ‘λ’ is the Faraday wavelength; ‘g’=9.8 m/s² is the gravitational acceleration constant; ‘σ’ is the surface tension; ‘ρₗ’ is the liquid density; and H=0.0015 m is the thickness of the liquid layer in the chamber. The kinematic surface tension σ/ρₗ=1.073×10⁻⁴ m³/s⁻² is a fitting parameter (R²=95.1%).

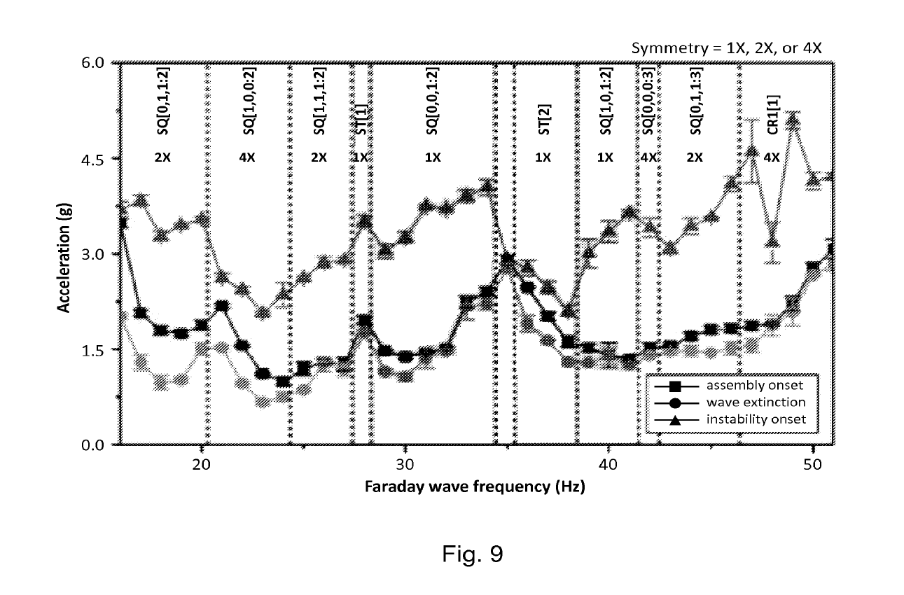

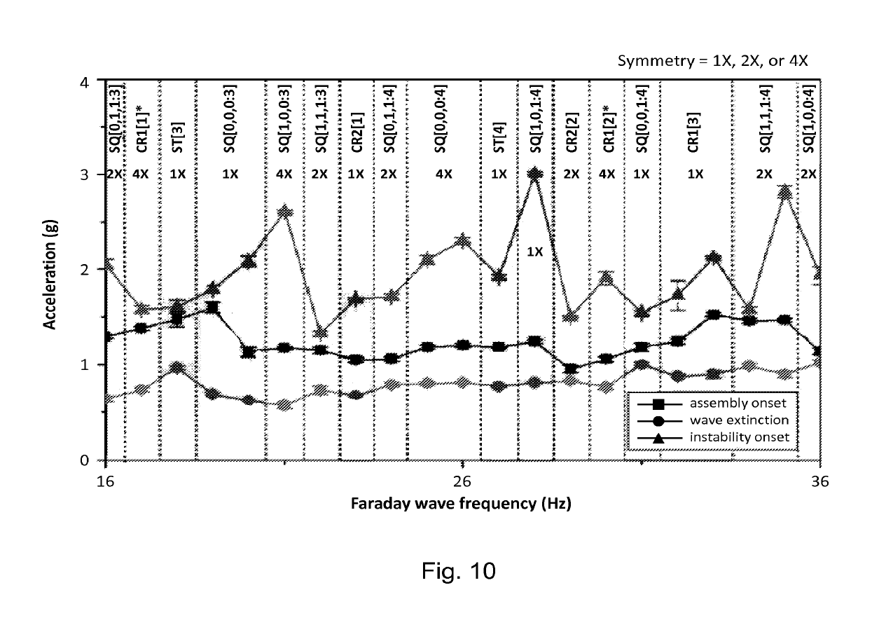

In one aspect, the diverse, assembled structures can be controlled through manipulation of the vibrational parameters. Phase diagrams were plotted as a function of the Faraday wave frequency and the vibrational acceleration to delineate the various assembled structures (FIGS. 9, 10). In the phase diagrams, the three data sets indicate assembly onset, wave extinction and instability onset. Bead assembly was observed to begin to take place after the formation of standing waves when the vibrational acceleration exceeded a threshold level (assembly onset). The amplitude of the standing waves increased with acceleration. When the acceleration exceeded a threshold level (instability onset), the assembled structure collapsed due to the standing waves exhibiting complex flow (i.e., going from vortices and chaotic waves to spills out from the chamber). The process of self-assembly stopped at acceleration levels that were typically lower than the onset acceleration level. This phenomenon can be explained by the hysteresis of the Faraday waves. Notably, the occurrence order of these assembled structures over frequency was the same and repeatable in 10 mm and 20 mm wide square chambers.

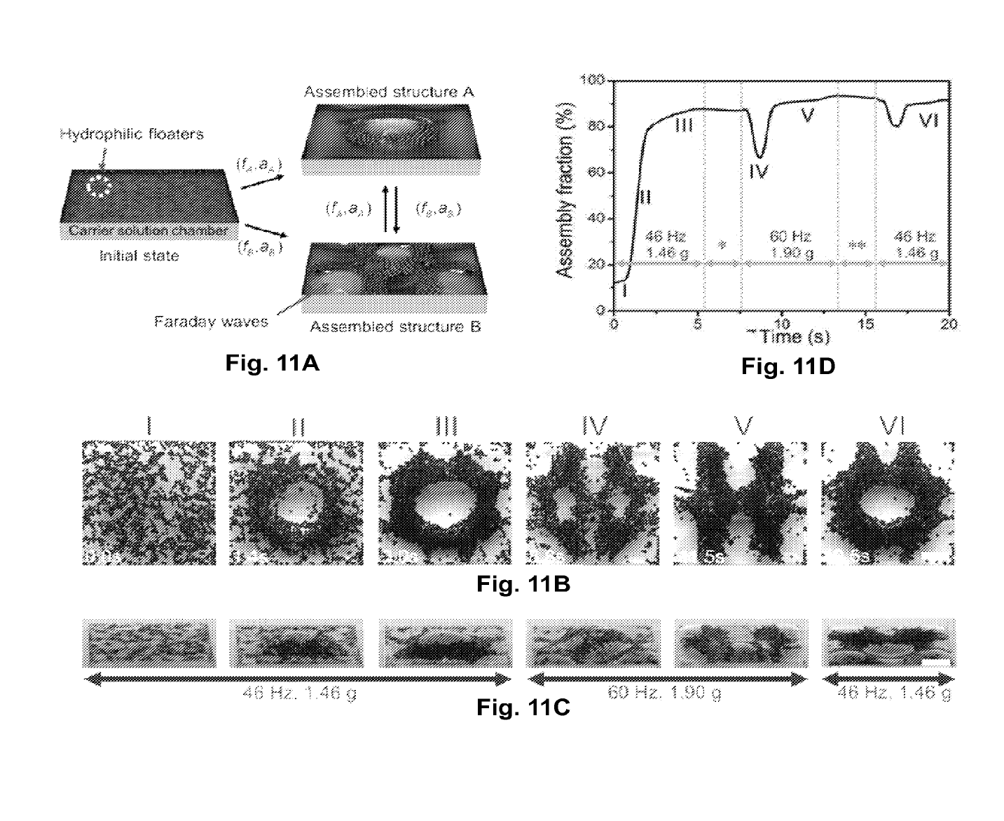

RAFT can be used to reconfigure the assembled structures to any of the other shapes on the phase diagrams by dynamically changing the vibrational parameters (FIG. 11A), which suggests that RAFT is a dynamic self-assembly process. A typical example of this dynamic self-assembly is demonstrated in FIG. 11B. Hydrophilic beads were assembled into a ring-shaped structure under 1.46 g at 46 Hz in a 10 mm wide square chamber. After the vibrational parameters were changed to 1.9 g at 60 Hz, the ring-shaped structure was transformed into an “H”-shaped structure. The assembled structure was restored to the ring shape after the vibration was set back to the original parameters. To characterize the fraction of beads utilized in the self-assembly, the assembly fraction, which is defined as the ratio of the number of assembled beads to the total number of beads in the chamber, was quantified. The results indicated that more than 90% of the beads were used in the self-assembly process (FIG. 11D).

A side view of the RAFT process is also depicted in FIG. 11C. Beads were observed to assemble as a monolayer on the nodal areas of the standing waves, leaving the antinodes uncovered. The antinodes of the standing wave alternated between crest and trough, which resulted in oscillations in the assembled structure after the self-assembly process reached a stable state. The topography of the standing wave lattice was varied by altering the vibrational parameters, which resulted in a redistribution of the beads in the chamber.

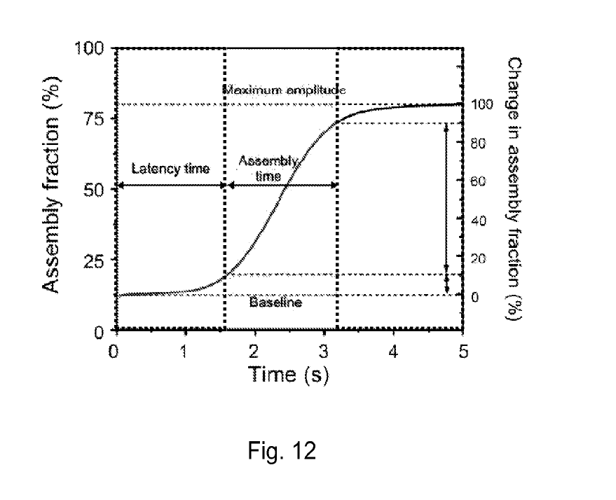

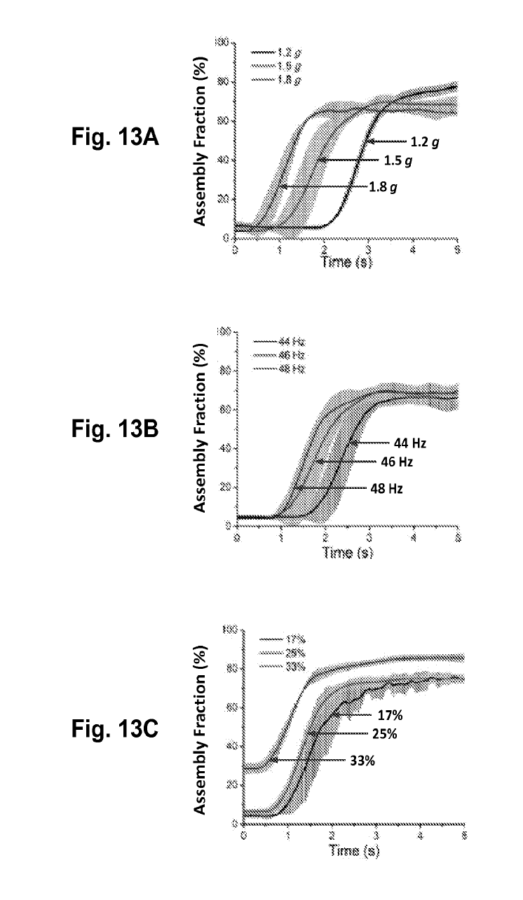

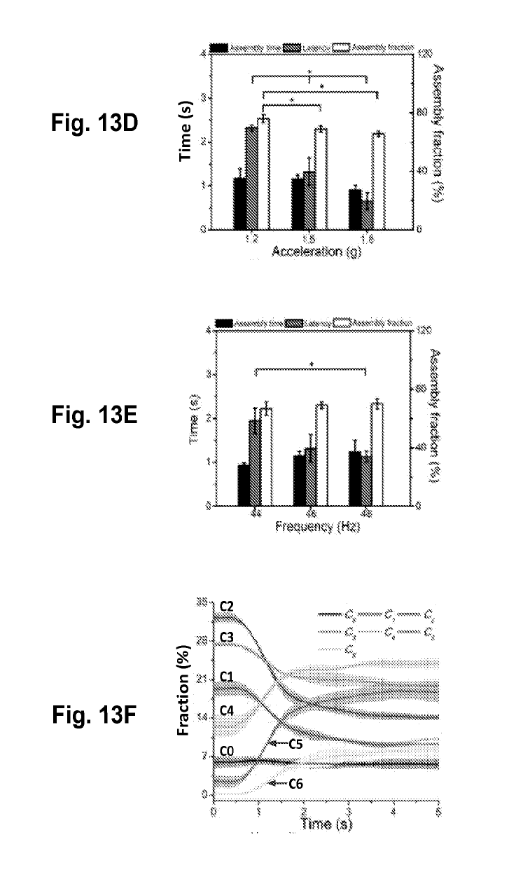

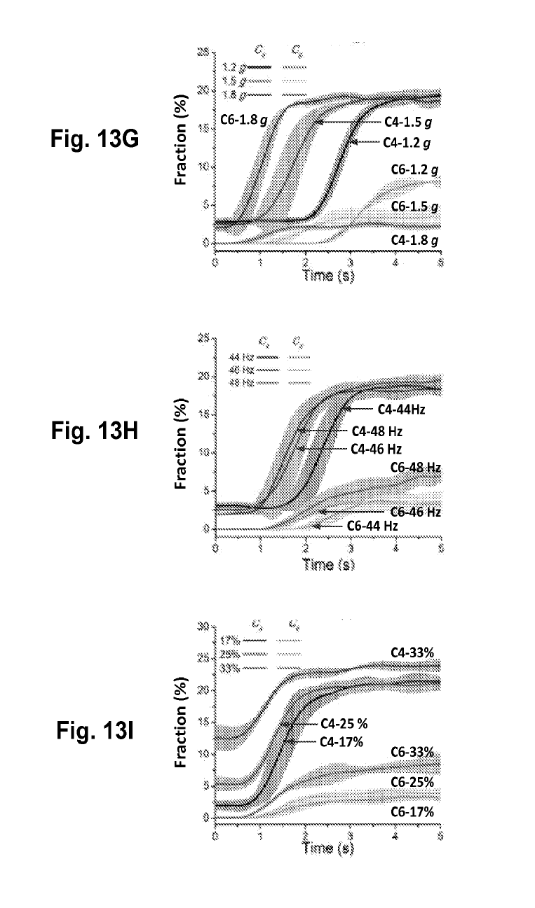

RAFT processes can be completed on the order of seconds. Specifically, self-assembly was observed to reach equilibrium in less than 5 seconds under varied driving accelerations and frequencies. In one aspect, more than 80% of the floaters are assembled within 3 seconds (FIG. 11D). The time evolution of the macroscopic structure (i.e., the global shape of the assembled structure) was quantified using the assembly fraction. A delay in the self-assembly is evaluated using latency time, which is defined as the duration from the application of the vibration to a 10% change in the assembly fraction, which can be evaluated as the duration required for a 10% to 90% change in the assembly fraction (FIG. 12). The effects of the vibrational acceleration and frequency on the self-assembly are illustrated in FIGS. 13A and 13B, respectively. Increased acceleration was observed to result in a significantly shorter latency time (FIG. 13D) without significantly changing the assembly time, and an increased frequency from 44 Hz to 48 Hz resulted in a significantly shorter latency time as well (FIG. 13E). Increasing the bead coverage fraction from 17% to 33% was also observed to result in an increase in the final assembly fraction without significantly changing assembly time and latency time (FIG. 13C).

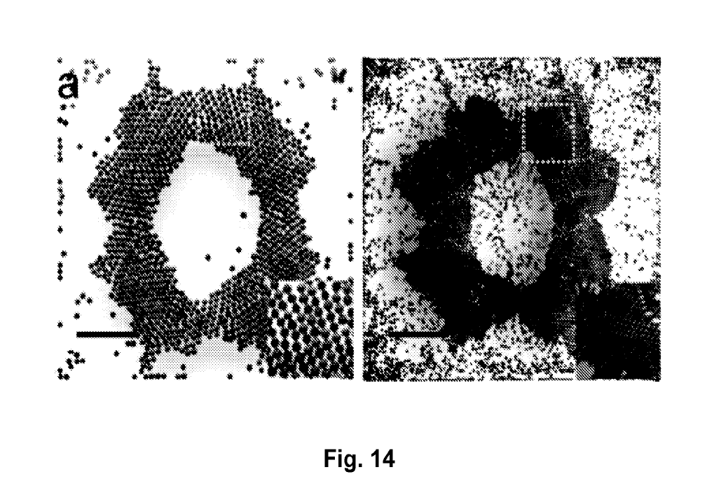

RAFT can be used to generate ordered particle arrangements (e.g., square close packing, hexagonal close packing) (FIG. 14) through an understanding of particle-to-particle interactions (i.e., lateral capillary interactions), which organize loosely packed particles into a close-packed structure. The time evolution of the microscopic structure (i.e., the bead arrangements within assembled structures) was quantified using the neighbor number (Ci), which is defined as the number (i) of closely packed beads surrounding a single bead (FIG. 15). In FIG. 13F, the fraction of C₁ to C₃ packing decreased, whereas the fraction of C₄ to C₆ packing increased during the self-assembly, which indicates that the self-assembly system minimized its potential energy by transforming its microscopic structure from a loose-packed to a close-packed structure. The C₆ fraction was observed to decrease when the acceleration was increased from 1.2 g to 1.8 g at 46 Hz (FIG. 13G). The fraction of C₆ structures increased as the vibrational frequency was increased from 44 Hz to 48 Hz at 1.5 g (FIG. 13H). However, the C₄ fraction was not observed to vary significantly when the vibrational acceleration and frequency increased (FIGS. 13G, 13H). Furthermore, an increase in bead coverage fraction from 17% to 33% resulted in an increased C₄ and C₆ packing fraction, which indicates a correlation between the formation of the microscopic structure (FIG. 13I) and the macroscopic structure (FIG. 13C).

In certain embodiments, the RAFT system exhibits unique dynamic self-assembly properties, including self-healing, self-adaptation and directional selectivity. Self-healing refers to the restoration of the original structure after perturbation. For example, a ring-shaped structure was generated under 1.95 g at 44 Hz and was physically broken by stirring with a pipette tip. When the stirring was terminated, the suspended beads restored the ring shape within a few seconds. Furthermore, the RAFT system acts as a self-adaptive system that can respond to a wide range of energy inputs by dynamically transforming the assembled structures. In one example, the assembled structure was a single cross at 1.95 g at 36 Hz, which spontaneously changed to four crosses when the vibration was adjusted to 2.41 g at 82 Hz. Self-healing along with self-adaptation is indicative that the RAFT system is robust with respect to changes in the energy input.

In addition, the direction of the assembled structures, which are generated by a one-fold symmetric standing wave lattice, can be manually rotated by 90° by being stirred with a pipette tip. Referring to FIG. 4B, this property can be explained by the observation that one-fold symmetric modes [0,1,0] and [0,0,1] (also modes [1,1,0] and [1,0,1]) can be overlaid with each other by a 90° rotation. As a result, one-fold symmetric modes can have the same potential energy. The directional selectivity suggests that the self-assembly system can behave as an energy-responsive binary switch.

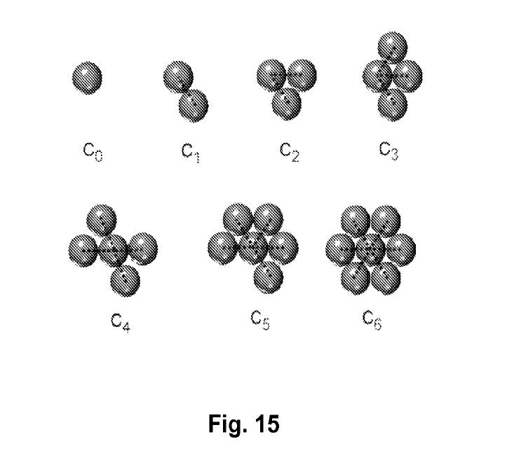

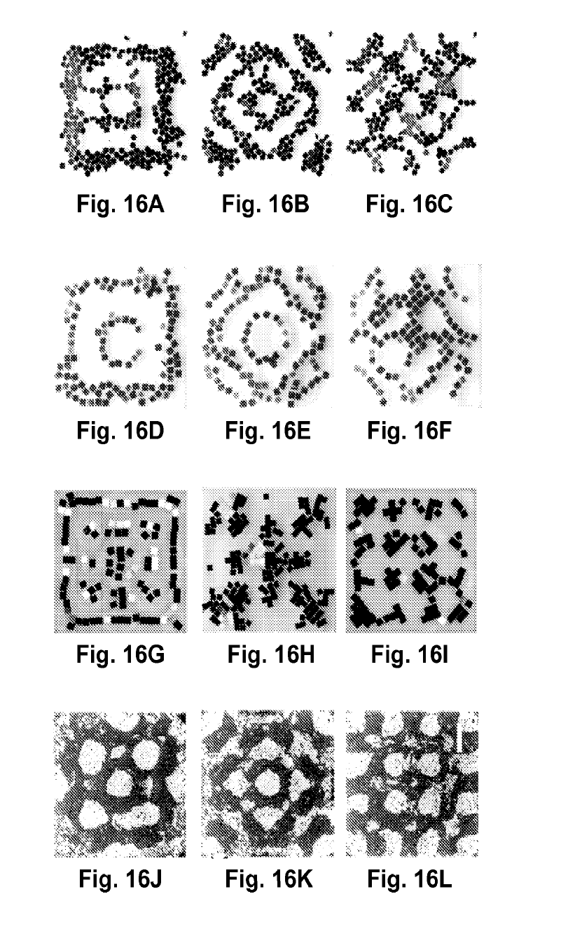

RAFT exhibits broad applicability to various materials, which can enable a wide range of real-world applications in different fields, such as tissue engineering, soft robots and microelectronics. In FIG. 16, assembly of a biocompatible and biodegradable biomaterial (e.g., gelatin methacrylate hydrogel (GelMA)), a soft material (e.g., polydimethylsiloxane (PDMS)) and microelectronic materials (e.g., silicon chiplets and copper powder) into nodal line patterns (FIGS. 16A-16F), antinode patterns (FIGS. 16G-16I) or complementary patterns (FIGS. 16J-16L) was demonstrated by exploring the wettability of these materials. In comparison with previous reports, RAFT enables controllable and repeatable generation of ten times more types of assembled structures with 10-100 times higher efficiency and speed.

Faraday waves are standing waves at the gas-liquid surface which originate from a parametric instability at the surface of a vertically vibrational liquid layer. A fluid layer perpendicular to the z-axis is driven by an acceleration of the form described by Eq. 4:

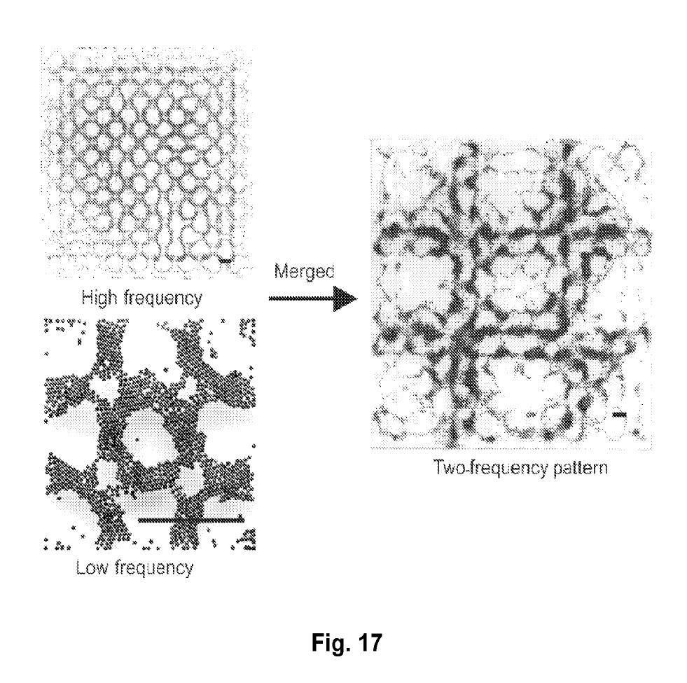

g(t′)=g+Aω² cos(ωt)=g[1+Γ cos(ωt)] (Eq. 4)

where ‘g(t)’ is acceleration on exerting on the surface, ‘g’ is the gravitational constant, ‘ω’ is the angle frequency of the driving force, and ‘A’ is the amplitude of vibrations. It is anticipated that RAFT can be used to generate arbitrary complex patterns with multi-scale structures by multiple-frequency forcing Faraday waves, as any periodic functions can be decomposed into the format of Fourier series. Preliminary result of templated self-assembly by a two-frequency forcing Faraday waves are demonstrated in FIG. 17.

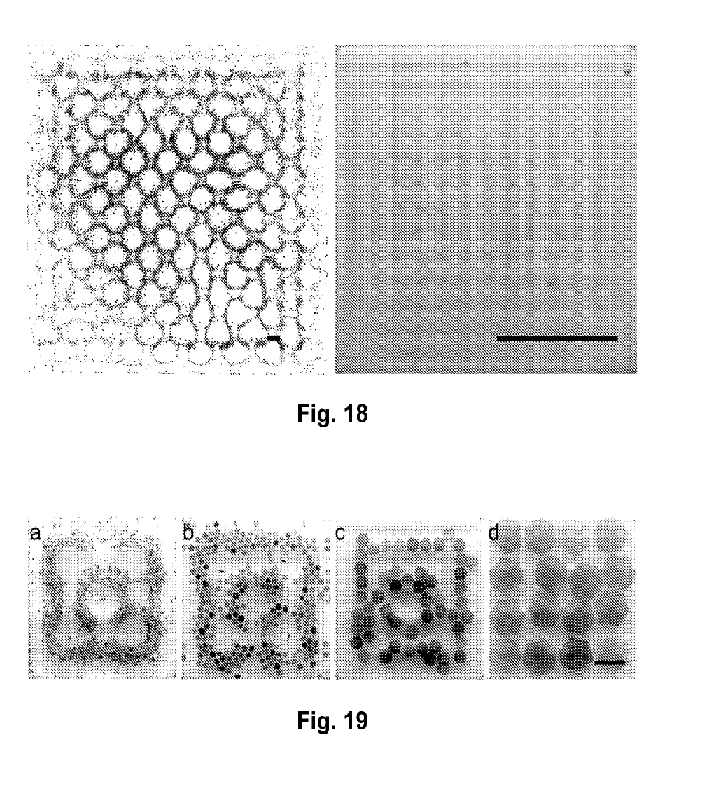

The scalability of RAFT was demonstrated in several different aspects. In a first aspect, it was demonstrated that the size of the chamber is scalable. RAFT can be performed in a square chamber with a side-length from about 5 mm to about 100 mm (FIG. 18). In a second aspect, it was demonstrated that the size of the assembled pattern is scalable. Identical patterns were generated in square chambers with a side-length of 10, 20 and 30 mm (FIG. 7). In a third aspect, it was demonstrated that floater size is scalable. RAFT was carried out with particles (floaters) of varied geometries with sizes ranging from about 500 μm to about 5 mm in diameter (FIG. 19). In certain embodiments, in order to best match the topography of standing waves, it is desirable to use floaters with a size 10 times less than the Faraday wavelength.

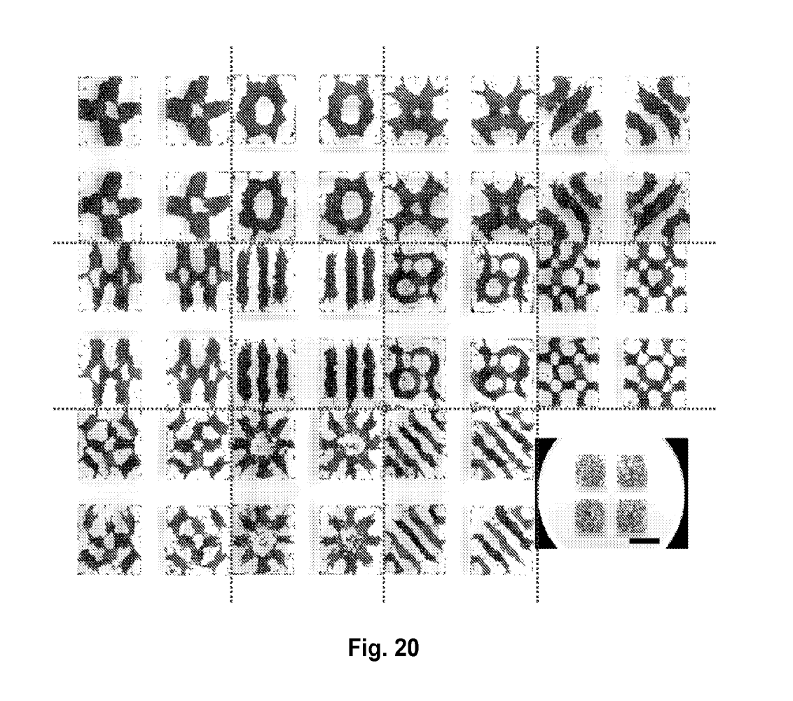

RAFT can be performed in a parallel format without the use of additional vibrational generators (FIG. 20). Simultaneous self-assembly of 200 μm beads into different patterns was demonstrated in a 2-by-2 chamber array. Parallel RAFT can be scaled up by providing additional chambers on a single device amounted on a single vibrational generator. Parallel RAFT holds a great promise for parallel manufacturing in real-world applications.

Following self-assembly with RAFT, a linking mechanism can be employed to permanently or temporarily join the self-assembled particles. The linking mechanism includes but is not limited to photo cross-linking, chemical cross-linking and thermo cross-linking. In some aspects, surface molecule recognition-based linking is employed in which floaters are coated with molecules, such as a nucleic acid or an antibody on their surface. Floaters can be specifically linked together during the assembly. In another aspect, geometric shape-based linking is employed in which floaters are fabricated with complementary structures, such as key and lock, or puzzle pieces. Floaters are linked together by geometric shape during the assembly. In one example of cross-linking, a photoactive hydrogel precursor solution containing gelation methacrylate, Irgacure 2959 can be used to link the particles as well as the working fluid. In one aspect, the cross-linking mechanism can provide a source of light such as UV light to affect the cross-linking process. In another example, a chemical cross-linking hydrogel precursor solution containing fibrinogen and thrombin can be used to link the particles as well as the working fluid. In this case, the cross-linking mechanism can effect a directed or disperse change in temperature or pressure.

The assembled structure described herein could be used for various applications in addition to those described in the examples below (see Appendix A). For example, the assembled structure can be used as a reconfigurable photomask for multiple photolithography for tissue engineering purposes. In this example, black carbon beads can be used as samples and are assembled into a predefined pattern. Once assembled, UV light will only pass through the area without carbon beads and cross-link the photo-resist or other photo cross-linkable hydrogel prepolymer solution.

In summary, the RAFT system has been demonstrated to enable self-assembly of micrometer-scale building blocks at the gas-liquid interface into a variety of ordered and symmetric structures using Faraday waves as a dynamic template has. RAFT provides new insights into scalable and parallel materials synthesis through global control of the emergent structures. The RAFT system can be used for studies on the unique phenomena of dynamic self-assembly, which potentially facilitates development of self-organizing and self-adaptive micro-robotic systems.

### EXAMPLE 1

### General Self-Assembly Techniques

Dyed polystyrene divinylbenzene beads with a diameter of 200 μm (Thermo Fisher Scientific, CA) were used as a prototypical hydrophilic sample in the experiments. The beads were uniformly dispersed onto a carrier solution that was composed of OptiPrep™ density gradient medium (Sigma, MO) mixed with Dulbecco's phosphate buffered saline (DPBS). To obtain the optimal assembly conditions, the carrier solution was adjusted using Tween 80 (Sigma, MO) to a final density of 1,060 kg m⁻³, a dynamic viscosity of 1.3×10⁻³ Pa-s and a surface tension of 50 mN m⁻¹. The carrier solution was enclosed in a square-shaped open chamber with a thickness of 1.5 mm. The suspension system was vertically vibrated by a vibration generator (U56001, 3B Scientific, Tucker, Ga.) that was further driven by a sine-wave signal provided by a function generator (HP 8116A, Hewlett-Packard GmbH, Germany). The vibrational acceleration and frequency were adjusted by the function generator. Acceleration was quantified by an accelerometer (MMA7341L, Freescale Semiconductor, TX). A high speed monochrome video camera (FASTCAM SA5, Photron, CA) was used to record the topography of the standing waves (side view) and to quantify the dynamic process of the bead assembly (top down view). Typical assembled structures were photographed with a digital SLR camera (A700, Sony, Japan). The images and data were analyzed and plotted using ImageJ (NIH, Bethesda, Md.), Matlab™ (MathWorks, MA) and OriginPro™ (OriginLab, MA).

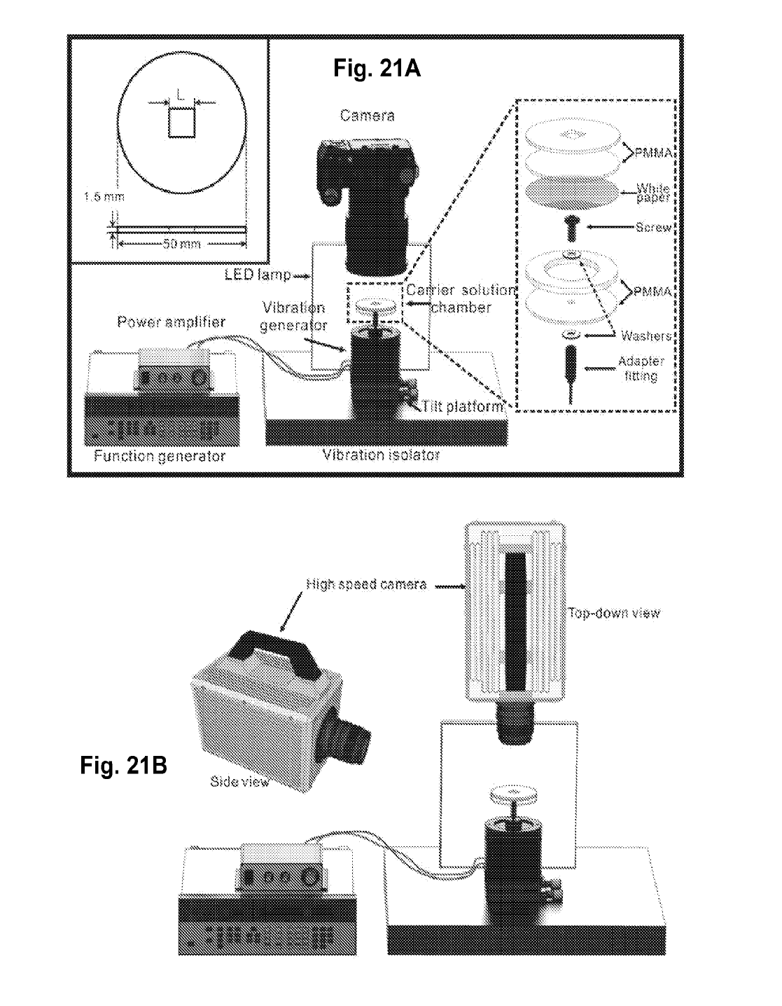

The experimental apparatus for implementing the RAFT system is illustrated in FIG. 21. Briefly, a vibration generator (U56001, 3B Scientific, Tucker, Ga.), driven by an audio amplifier (Lepai LP-2020A+, Parts Express, OH) and a function generator (HP 8116A, Hewlett-Packard GmbH, Germany), was used to generate vertical vibration. The vertical vibrational acceleration was validated as a sine wave by an accelerometer (MMA 7341 L, Freescale Semiconductor, TX). The vibration generator was fixed on a metric tilt platform (Edmund Optics, NJ), which was used to precisely adjust the level of the carrier solution chamber using a bubble level (Spirit Level, Hoefer, Mass.) as a reference. The metric tilt platform was fixed to a vibration damper (McMaster-Carr, GA) to prevent external perturbation. Square shaped carrier solution chambers with a thickness of 1.5 mm and side lengths of 10 mm and 20 mm were constructed from poly(methyl methacrylate) (PMMA) plates, double-sided adhesive (DSA) (iTapestore, Scotch Plains, N.J.) (not shown in FIG. 21A), and white paper, using a laser cutter (VersaLaser™, Scottsdale, Ariz.). The carrier solution chamber was mounted on the top of the vibration generator using an adapter fitting.

A digital SLR camera (A700, Sony, Japan) was used to record typical assembly structures using a 12 W LED as an illumination source (LitePad HO+, Rosco, CT) (FIG. 21A). A high speed monochrome camera (FASTCAM SA5, Photron, CA) was used to record the topography of the standing waves during floater assembly (side view) and to quantify the dynamic process of the floater assembly (top-down view) (FIG. 21B). A normal video camera (SDR-H 100 Camcorder, Panasonic, Japan) was used to record the unique properties of the Faraday-wave-directed self-assembly with a ring-shaped fluorescent lamp (150 W, 5400 K, Ardinbir Photography Studio) as a uniform illumination source.

The viscosity of the carrier solution was measured using a DHR3 rheometer (TA Instruments, DE, USA) with a stainless steel 1.1° cone plate with a diameter of 60 mm (Peltier plate acrylic). The surface tension of the carrier solution was measured using a Dataphysics DCAT II dynamic contact angle meter and tensiometer (Future Digital Scientific Co., NY, USA).

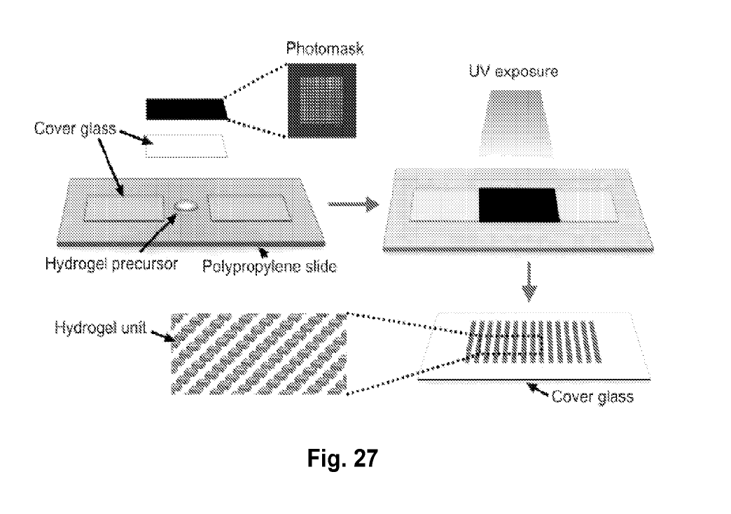

As depicted in FIG. 27, both gelatin methacrylate (GelMA) hydrogel units were fabricated by UV photolithography according to a previous published protocol (Xu, F. et al., Biomaterials 32, 7847-7855). GelMA powder was synthesized according to a previous published protocol (Soh, S., et al., The Journal of Physical Chemistry B 112, 10848-10853). To fabricate the microscale hydrogel units, the GelMA prepolymer solution was prepared by mixing (5% w/v) GelMA powder and 0.5% (w/v) photoinitiator (Igracure™ 2959; CIBA Chemicals) in Dulbecco's phosphate buffered saline (DPBS). The prepolymer solution was subsequently heated to 80° C. and vortexed to generate a homogeneous mixture. The prepolymer solution (30 μL) was pipetted onto a polypropylene slide. Two cover glasses (150 μm thick), which were used as spacers to define the thickness of hydrogel units, were adhered to the polypropylene slide. Another cover glass coated with 3-(trimethoxysilyl)propyl methacrylate (TMSPMA) (Sigma, MO) was placed on the spacer to spread the prepolymer droplet into a uniform thickness. A photomask with hexagonal patterns (1 mm in diameter) was designed using AutoCAD™ software (Autodesk, Inc., San Rafael, Calif.) and printed on transparent films using 32,000-dpi resolution (Fineline Imaging, CO). The photomasks were placed on the TMSPMA cover glass, which were then exposed to UV light (360-480 nm) at 138 mJ cm⁻² to cross-link the hydrogel precursor. After the UV polymerization process, the fabricated hydrogel units were adhered to the TMSPMA cover glass. The hydrogel units were washed with DPBS to remove excess prepolymer residue, stained with cibacron blue (Polysciences Inc, PA) and then washed with DPBS again to remove excess dye. The hydrogel units were stored in DPBS under ambient conditions prior to use in the experiments.

Silicon chiplets (1 mm×1 mm×0.1 mm) were fabricated from 2-inch silicon wafers (University Wafer, MA) using an automatic dicing saw (model DAD321, Disco Corp., Tokyo Japan).

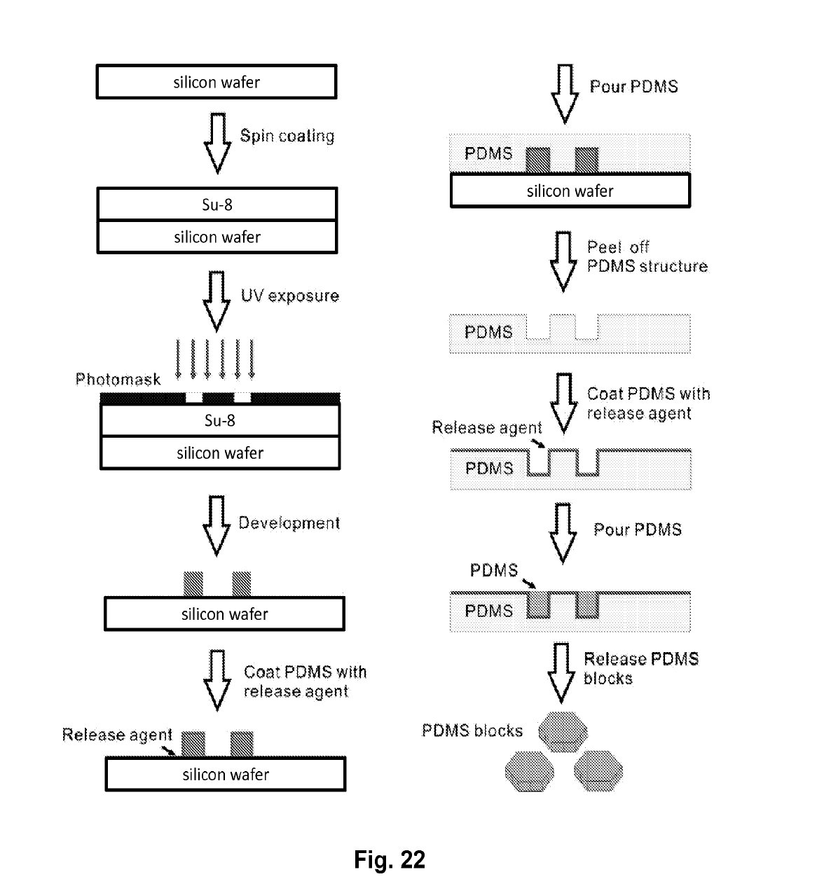

As illustrated in FIG. 22, polydimethylsiloxane (PDMS) building blocks were fabricated using photolithography and rapid prototyping with minor modifications (Duffy, D. C. et al., Anal. Chem., 70, 4974-4984, 1998). Briefly, an SU-8 master mold with a thickness of 500 μm was fabricated on a 4-inch silicon wafer using standard photolithography. Prior to use, the master mold was coated with a layer of trichloro(1H,1H,2H,2H-perfluorooctyl)silane (Sigma) to ease the release of the PDMS structure. The PDMS prepolymer was prepared by mixing the PDMS precursor and the curing agent in a ratio of 11:1. The PDMS mold was fabricated by curing the prepolymer on the master mold at 80° C. for 1 hour. Prior to use, the PDMS mold was coated with a layer of trichloro(1H,1H,2H,2H-perfluorooctyl)silane to ease the release of the PDMS blocks. The PDMS prepolymer for the building blocks was prepared by mixing the PDMS precursor and the curing agent in a ratio of 9:1. Sudan Red G (Sigma) was mixed with the prepolymer with a final concentration of 0.5% (w/w) to visualize the PDMS blocks. The prepolymer was poured onto the PDMS mold, and the excess prepolymer was removed with a razor blade. The prepolymer was cured at 80° C. for 1 hour in an oven. The final PDMS blocks were released by bending the PDMS mold.

Four types of beads with diameters of 10 μm (polystyrene, Invitrogen), 50 μm (polystyrene divinylbenzene, Thermo Scientific), 100 μm (polystyrene, Sigma) and 200 μm (polystyrene divinylbenzene, Thermo Scientific) were used in the experiments. All of the beads have hydrophilic surfaces. Prior to testing, the beads were washed with purified water (Milli-Q, Millipore Corporation, Billerica, Mass.) and the carrier solution chamber was washed with ethanol (Sigma) and dried using air blower. The beads were uniformly mixed in the carrier solution and then pipetted into the carrier solution chamber. The volume of solution was carefully adjusted to a brimful condition to avoid meniscus effects. The experiments were conducted immediately after the samples were loaded.

First, the carrier solution chamber was loaded with the carrier solution. The hydrogel units were sprayed with ethanol, detached from the TMSPMA cover glass using a razor blade, and subsequently transferred to the carrier solution chamber using a pipette tip. The hydrogel units were homogenously dispersed onto the surface of the carrier solution using ethanol; the dispersion process was induced by the Marangoni effect. The volume of the carrier solution was carefully adjusted to achieve a brimful condition prior to the experiments.

Silicon chiplets were washed with purified water and dried with nitrogen air. First, the carrier solution chamber was loaded with the carrier solution. Then silicon chiplets were manually dispersed onto the surface of the carrier solution. The silicon chiplets did not sink into the solution due to surface tension. The volume of the carrier solution was carefully adjusted to achieve a brimful condition prior to the experiments.

PDMS blocks were made hydrophilic by oxygen plasma treatment prior to the experiments. First, the carrier solution chamber was loaded with the carrier solution. Then, the PDMS blocks were manually dispersed onto the surface of the carrier solution. The volume of the carrier solution was carefully adjusted to achieve a brimful condition prior to the experiments.

Polystyrene divinylbenzene beads (200 μm) were uniformly mixed in the carrier solution and then pipetted into the carrier solution chamber. A copper-zinc alloy powder (60 mesh, Sigma) was uniformly dispersed onto the surface of the carrier solution. Copper-zinc powder didn't sink into the solution due to surface tension. The volume of the carrier solution was carefully adjusted to achieve a brimful condition prior to the experiments.

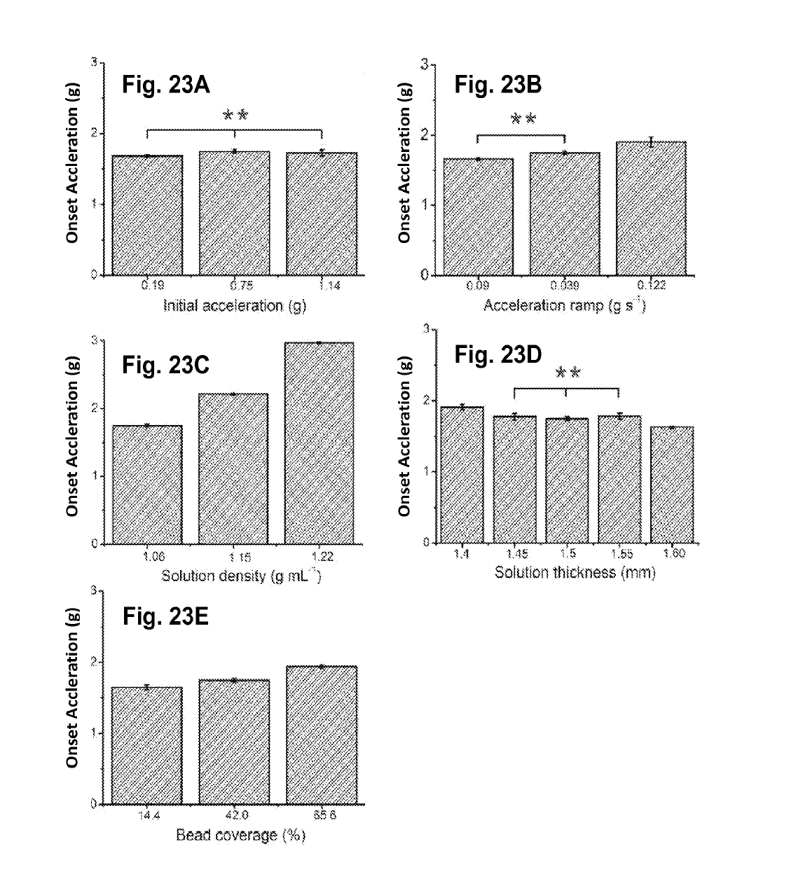

To control the self-assembled structures, the phase diagrams was explored as a function of the Faraday wave frequency (half the vibrational frequency) and the vibrational acceleration from which the assembly onset, wave extinction and instability onset were determined. Prior to testing, the effect of a series of experimental parameters on the assembly onset acceleration was investigated. Experimental parameters included the initial amplitude (initial acceleration) and the amplitude ramping rate (acceleration ramping rate) provided by the function generator as well as the solution thickness and density and the floater coverage (FIG. 23). It was observed that the initial amplitude did not significantly affect the onset acceleration and that the amplitude ramping rate significantly affected the onset acceleration when it was greater than 0.039 g s⁻¹. It was also observed that the onset acceleration increased with both increased solution density and increased bead coverage. There was no significant change in the assembly onset when the solution thickness was within 1.5±0.05 mm. Based on these observations, an amplitude ramp of 2 mV s⁻¹, an initial amplitude that was approximately 20 mV lower than the estimated assembly onset amplitude, a brimful condition (1.5 mm thick liquid layer), a solution density of 1.06 g mL⁻¹ and 42% bead coverage was used to explore the phase diagrams.

To determine the threshold conditions that led to assembly, the phase diagram was constructed using vibrational frequencies that ranged from 32 Hz to 102 Hz for the 10 mm×10 mm carrier-solution chamber and from 32 Hz to 72 Hz for the 20 mm×20 mm carrier-solution chamber. A sampling rate of 2 Hz was used for both phase diagrams. The carrier solution was used in the experiments with a bead coverage rate of 42%. The assembly onset was defined as the acceleration of the carrier-solution chamber at which lateral movement of the beads for assembly were first observed. The instability onset was defined as the acceleration of the chamber at which the assembled structure was destroyed due to chaotic flow, vortex formation, solution spill-out from the chamber or a combination of these events. The wave extinction threshold was defined as the acceleration of the chamber at which vertical vibration of the standing waves could no longer be observed during the decreasing acceleration.

An accelerometer was employed to measure vertical acceleration of the carrier-solution chamber and correlate the acceleration with the driving voltage amplitude from the function generator.

A high-speed camera (Photron SA5) was used to study the dynamic behavior of the floater assembly. Videos of the assembly process were recorded at 1000 fps. ImageJ (NIH, Bethesda, Md.) was used to extract the coordinates for each floater. The floaters were assigned to groups based on their spatial proximity to each other. Two floaters were defined to be connected when the distance between them was less than 30 μm. The connected floaters were assigned to a unique group. The macroscopic structure of assembled patterns was characterized by the assembly fraction, which was calculated as the ratio of the number of floaters in the largest group to the total number of floaters in the chamber. The microscopic structure of the assembled patterns was characterized by neighbor number (Cᵢ, where i is the number of neighbors connected to each floater and ranges from 0 to 6). The Cᵢ fraction is calculated using the ratio of the number of floaters with i surrounding neighbors (distance between them was less than 30 μm) to the total number of floaters in the chamber. The assembly fraction and neighbor number were calculated for each frame in the videos obtained from the high-speed camera. The data from this study were reported as the means±standard deviation. For each study, the assembly fraction and neighbor number were averaged over three videos, and the final curves were smoothed by taking the average of 20 adjacent points.

Generation of standing wave lattice: Stable standing waves were generated in a lattice format in a low-viscosity fluidic system (Gollub, J. P. et al., Physica D: Nonlinear Phenomena 6. 337-346), where arrangement of standing waves was dependent on the chamber's geometry due to side-wall boundary effect. The side-wall boundary effects is effective in the decay length as described by Eq. 5:

l_{d}≈σ/(4νρω) (Eq. 5)

where ‘σ’ is the surface tension; ‘ν’ is the kinematic viscosity; ‘ρ’ is the liquid density; and ‘ω’ is the circular driving frequency. The side-wall boundary effect can be neglected only when L>>l_{d}, where ‘L’ is the side length of the square chamber. In our system, l_{d} is approximate to 0.03 m, when driving frequency is 50 Hz.

### EXAMPLE 2

### Self-Assembly of Microscale Cell-Encapsulating Hydrogel Units

The effect of acceleration on cell viability was investigated for cell encapsulating hydrogel units. The results indicated more than 80% cell viability for 30 seconds with vertical acceleration up to 3 g.

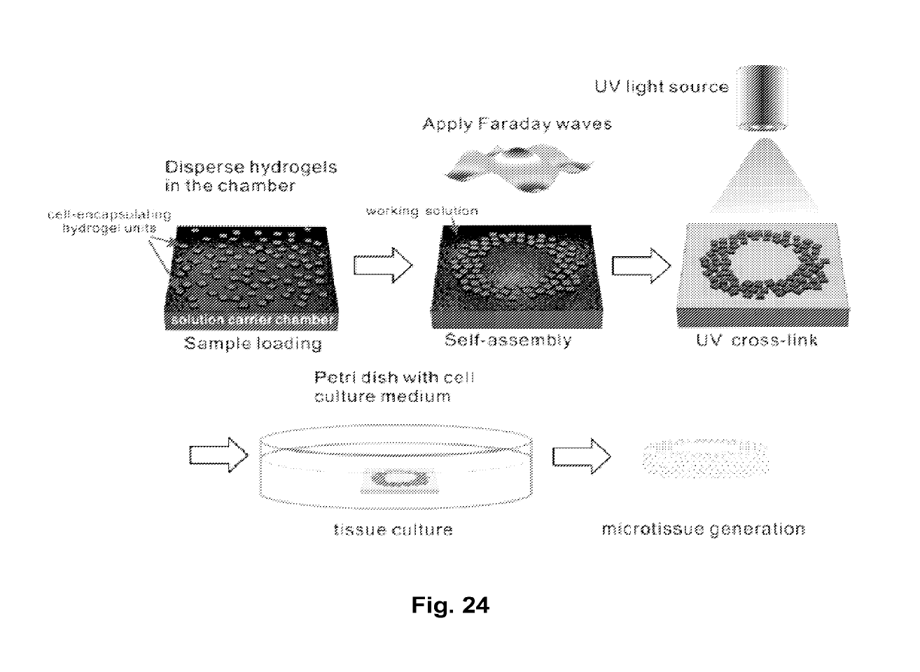

Assembly of cell encapsulating hydrogel units with RAFT is depicted in FIG. 24. One general assembly approach begins with dispersion of the hydrogel units at a gas-liquid interface in a solution carrier chamber. Then, Faraday waves can be generated at the gas-liquid interface by vertically oscillating the chamber. The hydrogel units can assemble at nodal lines of the standing waves, at which time vertical vibration can be stopped. The assembled pattern can be exposed to UV light in order to fix the pattern in the chamber by cross-linking the working solution in the chamber. The cross-linked pattern can then be transferred to a cell culture dish filled with cell culture medium for tissue generation. The cross-linked hydrogel formed by UV irradiation of the working solution can be formulated to degrade on a shorter time-scale than the microscale hydrogel units. As a result, microtissues can be cultured in the shape of the RAFT pattern.

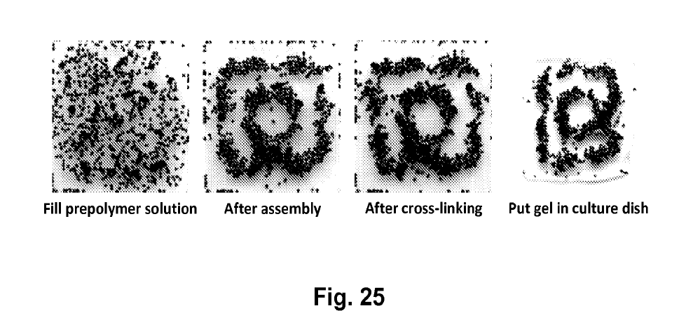

To experimentally demonstrate the above principle, GelMA hydrogel units were stained with cibacron blue for visualization in the working solution. FIG. 25 illustrates an example of hydrogel units that were cross-linked and transferred to a cell culture dish.

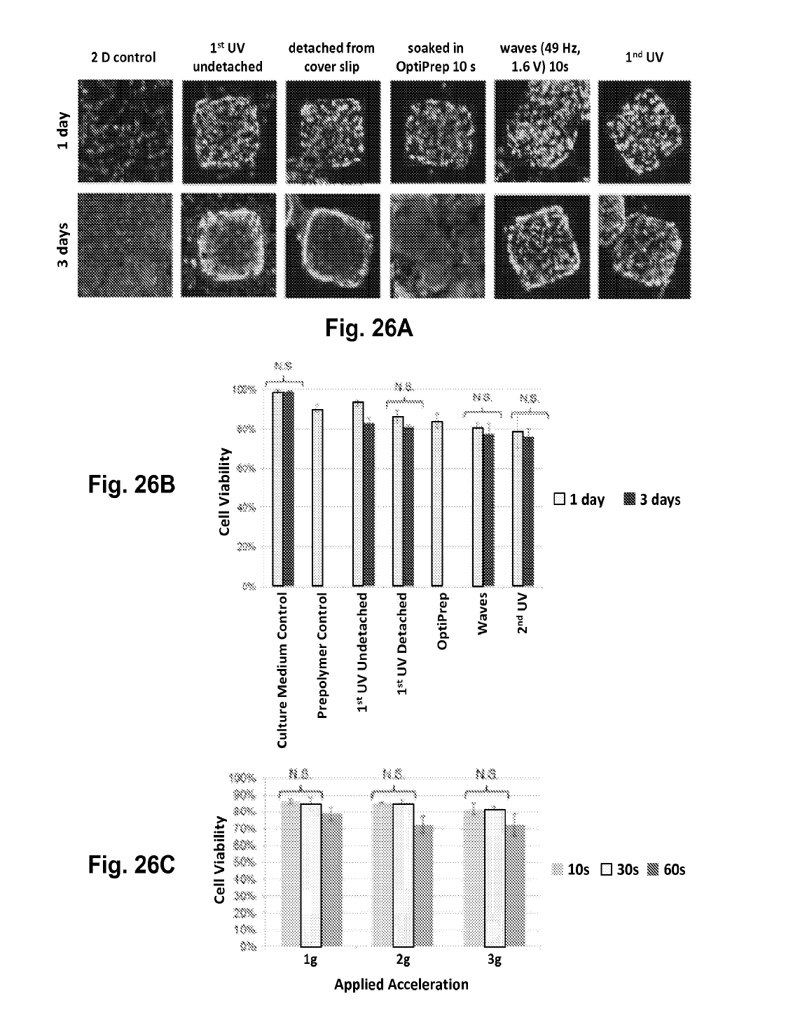

Cell viability test live/dead assays were performed to investigate the effect of Faraday waves on cell viability. Cells encapsulated in hydrogel units were exposed to Faraday waves at 49 Hz and 1 g for 10 s, which was enough time to allow for completion of the self-assembly process. FIG. 26A depicts an example of the live/dead staining assay after one day and three days of cell culture. The three day cell viability assay for the Faraday wave treated group remained nearly constant (FIG. 26B), which is indicative that the Faraday wave treatment has no long-term adverse effects on cell viability. The effect of Faraday waves on cell viability under various treatment time and acceleration was also investigated (FIG. 26C). The results indicated an insignificant difference when treatment time was less than 30 s for various accelerations.

NIH 3T3 mouse fibroblast cells were cultured in DMEM (Gibco) supplemented with 10% (v/v) NCS (Gibco), 100 units mL⁻¹ penicillin, and 100 μg mL⁻¹ streptomycin, at 37° C. in a 5% CO₂ atmosphere. Once cells grew to 90% confluence, cells were washed with buffered saline (PBS, pH 7.2) for three times to eliminate debris, detached from the substrate by treatment with Trypsin-EDTA solution (Gibco), centrifuged at 1000 rpm for 5 minutes, and then resuspended in cell culture medium. Cell concentrations were determined with a hemocytometer and adjusted to 4 million mL⁻¹ prior to further use.

Microscale cell encapsulating hydrogel units were fabricated with Gelatin methacrylate (GelMA) hydrogel by UV photolithography according to a previous published protocol (FIG. 27) (Xu, F. et al., Biomaterials 32, 7847-7855, 2011). GelMA prepolymer powder was synthesized according to a previous published protocol (Nichol, J. W. et al., Biomaterials 31, 5536-5544, 2010). To fabricate the microscale hydrogel units, the GelMA prepolymer solution was prepared by mixing (5% w/v) GelMA powder and 0.5% (w/v) photoinitiator (Igracure™ 2959; CIBA Chemicals) in Dulbecco's phosphate buffered saline (DPBS). The prepolymer solution was subsequently heated to 37° C. and vortexed to generate a homogeneous mixture. Cell suspension solution was gently aspirating with a pipette, and mixed with the GelMA prepolymer solution a volume ratio of 1:1 to generate a final cell concentration of 2 million mL⁻¹. The mixed solution (30 μL) was pipetted onto a polypropylene slide. Two cover glasses (150 μm thick), which were used as spacers to define the thickness of hydrogel units, were adhered to the polypropylene slide. An additional cover glass coated with 3-(trimethoxysilyl)propyl methacrylate (TMSPMA) (Sigma, MO) was placed on the spacer to spread the prepolymer droplet into a uniform thickness. A photomask with hexagonal patterns (500 μm in diameter) was designed using AutoCAD™ software (Autodesk, Inc., San Rafael, Calif.) and printed on transparent films using 32,000-dpi resolution (Fineline Imaging, CO). The photomasks were placed on the TMSPMA cover glass, which were then exposed to UV light (360-480 nm) at 500 mJ cm⁻² (37 mm height, 18 s) to cross-link the hydrogel prepolymer. After the UV polymerization process, the fabricated hydrogel units were adhered to the TMSPMA cover glass. The hydrogel units were washed with DPBS to remove excess prepolymer residue. The hydrogel units were detached from the cover glass by razor blade and used immediately. All the operations were performed in UV sterilized laminar flow hood.

A Faraday wave working solutions for tissue engineering was composed of 10% (w/v) PEGDMA 1000, 1% (w/v) photo-initiator (Igracure™ 2959; CIBA Chemicals), 0.01% (v/v) Tween and 18% (w/v) iodixanol in PBS.

For each cell viability test, one cover slip of hydrogel units were transferred to the chamber filled with OptiPrep-DPBS (1.06 g mL⁻¹). Cell encapsulating hydrogel units were exposed to Faraday waves under different accelerations and times. Then the hydrogel units were transferred to a 48 well plate for further cell culture. Cell viability tests were performed after one day's cell culture. All the experiments were performed in a UV sterilized laminar flow hood.

Cell staining solutions were prepared by dissolving 20 μL ethidium bromide and 5 082 L calcein (LIVE/DEAD® Viability/Cytotoxicity Kit, Invitrogen, L-3224) in 10 mL DPBS. 200 μL of cell staining solution were added to each well after complete removal of cell culture medium. Cells were then transferred to an incubator for 15-minute incubation. Cells were prepared for microscopy by replacing the cell staining solution with 600 μL of DPBS in each well.

Microscopy was performed using an Axio Observer D1 inverted microscope (Zeiss) equipped with a CCD camera (AxioCam NRM, Zeiss). Image processing and cell counting were performed with ImageJ (NIH).

### EXAMPLE 3

### Three Dimensional Engineering of Tissue Functional Units

In another aspect, the aforementioned RAFT system (FIG. 28) might be applied to 2D engineering of tissue functional units. The topography of the gas-liquid interface can be used as a template, which can be parametrically controlled by frequencies and amplitudes of Faraday waves. Microscale hydrogel units can specifically be assembled on a liquid template into a monolayer structure, which can be further stacked layer by layer into a 3D architecture. By assembling cell-encapsulating hydrogel units into 3D constructs and culturing them for further maturation, hydrogel scaffolds are can degrade and be completely replaced by cellular growth and extracellular matrix (ECM) deposition, resulting in formation of 3D native like tissue constructs. The LOC technologies can be developed to interface with the engineered tissue functional units and provide a simulated microphysiological environment for further tissue functionalization. In vivo evaluation of an engineered graft can be performed with ectopic implantation in a mouse model.

The RAFT system can be applied to the area of tissue engineering in a number of ways. In a first aspect, RAFT can be used to construct repetitive complex 3D zonal architectures from microscale hydrogel units. In a second aspect, RAFT can be used to engineer 3D tissue functional units. In a third aspect, RAFT can be used to evaluate tissue functionalization and characterization both in vitro and in vivo.

RAFT can provide a technological platform and related procedures by integrating templated self-assembly technologies, microscale hydrogel technologies and LOC technologies for high-throughput 3D engineering of tissue functional units. This platform can become a broadly available biotechnological tool that could be applied for many fields such as tissue engineering, regenerative medicine and preclinical drug screening.

Significance of Tissue Engineering: Tissue engineering explores harmony of cells, engineering, materials sciences, and biochemical factors to repair an injury or replace the function of a failing tissue/organ. Tissue engineering holds great promise to improve the healthcare and life quality of millions of people worldwide by innovating paradigms for trauma, disease therapies, diagnostics and drug discovery. In vivo, cells are embedded in a 3D microenvironment composed of ECM and neighboring cells with a defined spatial distribution. Tissue functionality is supported by these components and influenced by their relative spatial interactions and locations. When cells are cultured in two-dimensional (2D) monolayers, they display significant differences in gene expression compared with cells in native tissues and in 3D culture conditions. Hence, 2D systems do not effectively represent the complex 3D tissue environment. Tissue engineering approaches therefore focus on design and generation of 3D tissue constructs to mimic biological, chemical and physical properties of native tissues, such as cell types and functionalities, physiological environments and material properties.

Significance of Tissue Functional Units: An organ is a collection of tissues joined together to serve a common function. Mesoscale tissue functional units exist between single-cell scale and organ scale. Most of these tissue functional units are well vascularized 3D structures with characteristic dimensions of a few hundred micrometers in diameter and several millimeters in length. Such dimensions facilitate oxygen exchange and nutrient/waste transfer. Tissue functional units usually consist of several types of cells that collaborate to carry out functions for the corresponding organ. For example, the hepatic acinus is a basic unit that carries out metabolic functions in the liver. To engineer the complex tissues of the organs, bottom-up technologies that enable generating large amount of repeating structures are urgently required.

Hepatic Acini in Liver: The liver plays an important role in metabolisms, including decomposition of red blood cells, plasma protein synthesis and detoxification. Each year, approximately 30,000 deaths are caused by acute and chronic liver failure in the United States. Liver transplantation is the only therapeutic solution for end-stage liver disease, whereas the availability of liver donors is between 6,000 and 7,000 each year. Therefore, technologies that facilitate the engineering of partial hepatic tissues or the whole liver are desirable for liver transplantation.

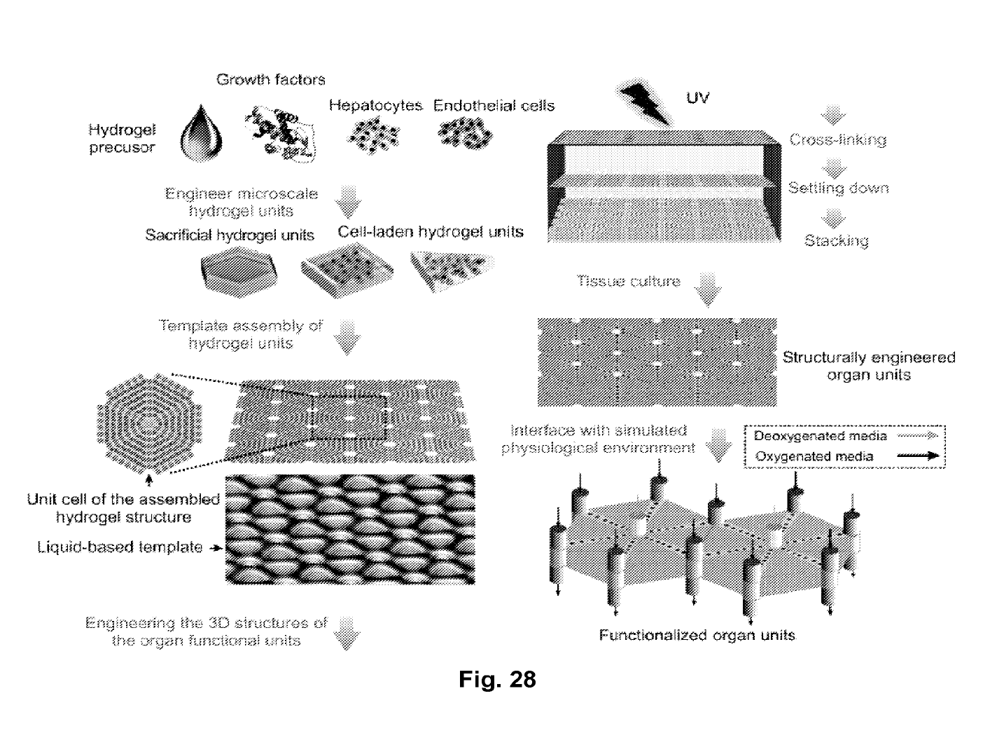

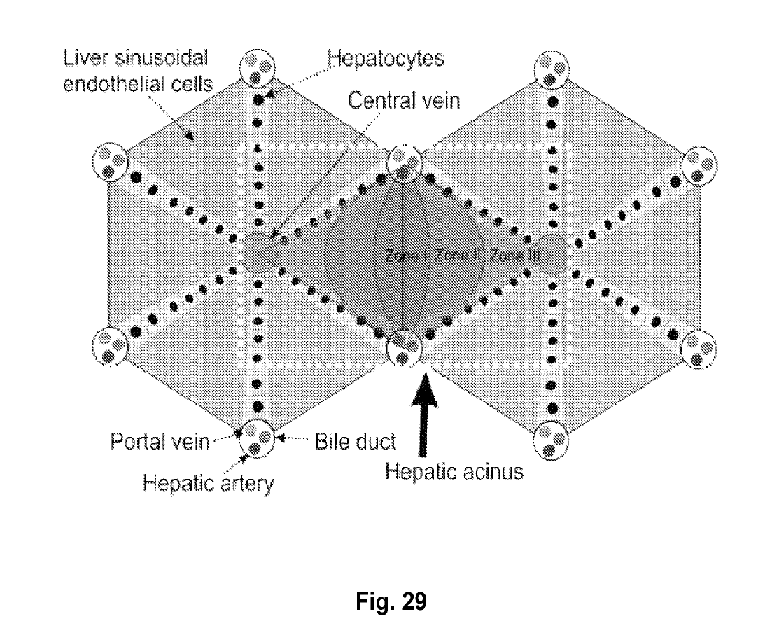

The hepatic acinus is a functional unit of the liver. The acinus consists of an irregularly shaped, roughly ellipsoidal mass of hepatocytes aligned around the hepatic arterioles and portal venules just as they anastomose into sinusoids. The acinus is roughly divided into three zones that correspond to the distance from the arterial blood supply (FIG. 29). Hepatocytes in zone I are specialized for oxidative liver functions such as gluconeogenesis, β-oxidation of fatty acids and cholesterol synthesis, while hepatocytes in zone III support glycolysis, lipogenesis and cytochrome P-450-based drug detoxification. This specialization is reflected in bio-chemical make-up of the cells. For example, zone III cells have a high concentration of CYP2E1 and thus are highly sensitive to NAPQI production in acetaminophen toxicity. These properties can be utilized as criteria in the functional validation of engineered hepatic acini.

Microscale Assembly Technology: Microscale assembly technologies are utilized in bottom-up tissue engineering approaches. Such technologies focused on mimicking microscopic structures of native tissues (i.e., repeating units) and building tissue scaffolds from cell-encapsulating assembly blocks (e.g., microscale hydrogel units). Microscale assembly technologies allow rapid generation of tissue scaffolds by employing interactions among building blocks (i.e., self-assembly) or between building blocks and external agitation fields (i.e., directed assembly). Self-assembly determines the microscopic structure (i.e., arrangement of building blocks) of the assembled scaffolds, while directed assembly determines the global structure of the assembled scaffolds. A number of microscale assembly technologies have been recently developed to build hydrogel scaffolds for tissue engineering purposes. Example technologies include acoustic assembly, magnetic assembly, capillary force based assembly, molecular recognition, and shape recognition. Despite the potential of these technologies for tissue engineering, some unaddressed challenges can limit their widespread applicability. Such challenges include generation of repeating 3D zonal architectures, microscale resolution and throughput for complex architectures (Table 1, Performance comparison of reported bottom-up tissue engineering technologies and developed Faraday wave-based templated self-assembly technology). To fulfill the practical applications of tissue engineering, assembly technologies that can meet these challenges are greatly needed.

TABLE 1

| | | | | Surface | |
|---|---|---|---|---|---|
| | Molecular | Railed | Geometrically | Tension | |
| | Recognition | Microfluidics | Docking | Assembly | RAFT |
| Throughput | Low | Low | High | Medium | High |
| Block Size | N/A | 10-1000 | 50-500 | 50-500 | 10-5000 |
| (μm) | | | | | |
| Structure | Low | High | High | Low | High |
| Complexity | | | | | |
| Adverse | Medium | Low | Low | Medium | Low |
| Effects on Cells | | | | | |
| Assembly | 20-120 s | Scales with # | ~20 min | ~60 s | 5-10 s |
| Time | | of blocks | | | |

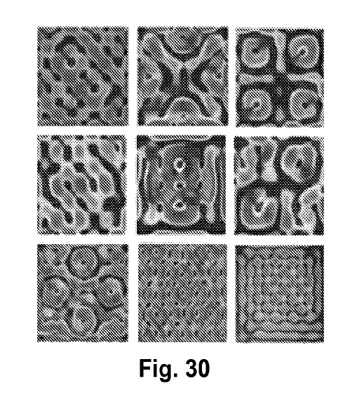

Faraday wave-based templated self-assembly technology: Faraday waves are standing waves at the gas-liquid surface that originate from a parametric instability at the surface of a vertically-vibrated liquid layer. By controlling the vibrational parameters (i.e., the frequencies and the accelerations), basic waveforms such as stripes, squares, triangles and hexagons (FIG. 30) can be obtained. These waveforms can then be tailored and combined into highly-diverse periodic topographies at the gas-liquid interface in a controllable manner. The topography resolution is determined by the wavelength of Faraday waves and ranges from tens of microns to a few millimeters. Composite topography of the gas-liquid interface can be designed by multiple-frequency forced Faraday waves. By using the generated topography as a template, microscale hydrogel units can be specifically assembled on the liquid template into zonal structures.

Biomaterials: Hydrogels have been used extensively as scaffolds for tissue engineering due to their moldability, high water content, high porosity, and biocompatibility. Owing to these features, cell-encapsulating hydrogels can be used as building blocks for constructing 3D tissue structures in the bottom-up tissue engineering. The native cell microenvironment is highly complex in terms of biological, chemical and physical properties. Natural and synthetic polymers such as hyaluronic acid, collagen, fibrin, alginate and polyethylene glycol have been used to mimic this complexity. There is a significant need for novel hydrogel engineering technologies that enable engineering microscale hydrogel units in multiple aspects (e.g., geometry, surface wettability, degradation rate, porosity, bioactive molecules, cell type and density) to mimic the native cell environment composed of ECM and neighboring cells with a defined spatial distribution.

Lab-on-a-Chip Technologies: “lab-on-a-chip” describes device miniaturization, integration and automation at the micro- and nano-scale across diverse disciplines (e.g., chemistry, biology, bioengineering, and biomedical engineering). Device miniaturization brings a series of benefits including, but not limited to: (i) low sample consumption, (ii) faster analysis, (iii) shorter response times, (iv) improved process control, and (v) massive parallelization. Owing to these benefits, LOC technologies provide a great platform for maximal control of physical, chemical and biological factors on chip-based microenvironments (e.g., chemical gradient, flow rate, oxygen concentration, and temperature) for broad tissue engineering applications. However, interfacing engineered tissue units with LOC system remains challenging due to the small size of capillary blood vessels (10-40 μm in diameter) and control fluid perfusion within these capillaries. Hence, there is significant need for novel LOC technologies that enable seamlessly interfacing with capillary blood vessels in the tissue units and precise control over fluid flow in the capillaries.

The RAFT system can be applied to the integration of multiple converging fields and technologies. First, RAFT technology can address limitations of current microscale self-assembly technologies for engineering 3D tissue construct with zonal architectures. RAFT enables dynamically configuring the topography of liquid templates by adjusting the vibrational parameters of Faraday waves (i.e., forcing frequencies and accelerations) in a parallel manner. Any multiscale periodic template can be designed and created by multiple-frequency forced Faraday waves. The self-assembly can be simultaneously performed in standard cell-culture consumables that are compatible with current cell culture techniques. The self-assembly can be typically be completed within 10 seconds independent of the area of the assembled structure. In one aspect, building blocks with sizes ranging from 0.1 μm to about 1 m, and ore preferably, about 10 μm to about 5 mm can be specifically assembled to defined positions of standing waves. Second, smart hydrogel engineering technologies can be used to engineer the various aspects of microscale hydrogel units (e.g., geometry, surface wettability, degradation rate, porosity, bioactive molecules, cell type and density) for multiple purposes at the same time. Third, novel LOC technologies can be implemented to interface with the engineered tissue functional units, to mimic in-vivo microphysiological environments for further maturation. Overall, a technological platform and tissue engineering approaches can be implemented for the generation of vascularized 3D tissue functional units by merging microscale assembly technologies (RAFT), LOC technologies, biomaterials sciences and tissue engineering principles.

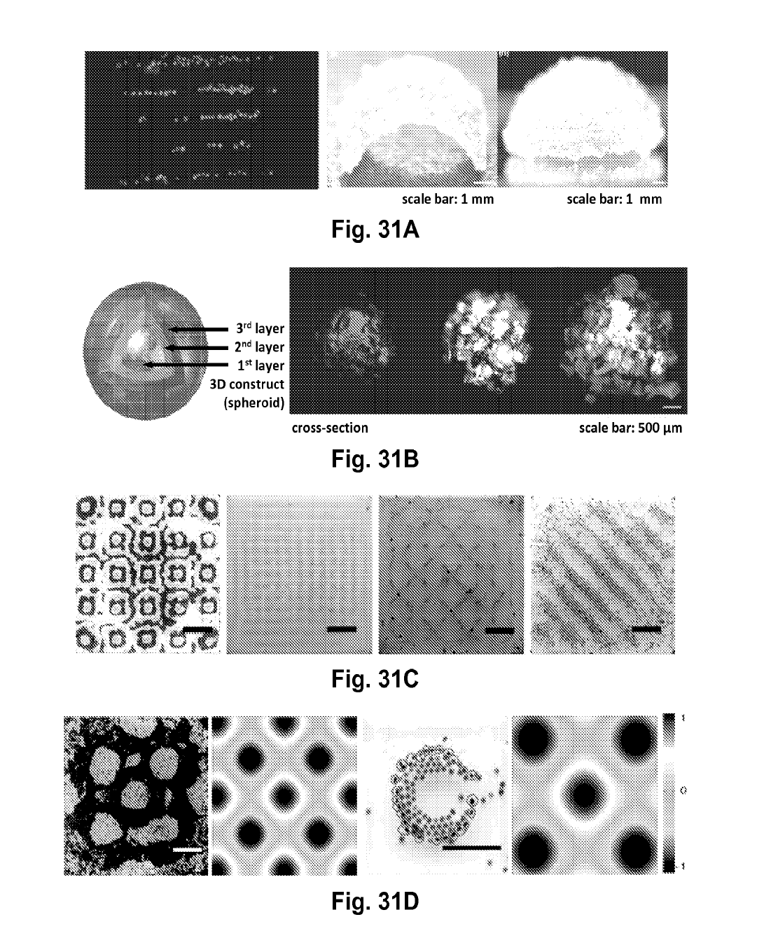

Microscale Assembly Technologies: Referring to FIGS. 31A-31D, diverse microscale assembly technologies for building 3D tissue constructs from microscale cell encapsulating hydrogel units have been developed (Xu, F. et al., Biomaterials 32, 7847-7855; Tasoglu, S. et al., P Adv. Mater. 25, 1137-1143; Gurkan, U. A. et al., Adv. Mater. 25, 1192-1198; Tasoglu, S., et al., J. Tissue Eng. Regen. Med. 6, 224-224; Xu, F. et al., Adv. Mater. 23, 4254-4260; Park, J. H. et al., BiotechnoL Bioeng. 106, 138-148; Moon, S. et al., Tissue Eng. Part C-Methods 16, 157-166). For instance, in the acoustic field directed assembly, microscale hydrogel units with different sizes and shapes (e.g., cubes, lock-and-key shapes, tetris, saw) were concentrated through the application of acoustic fields and assembled together by shape recognition. Assembly of multilayer hydrogel units was demonstrated by layer-by-layer stacking technique. Cell viability in hydrogel units was over 93% after acoustic agitation. In another study, magnetic fields were used in the directed assembly of magnetic nanoparticle encapsulated microscale hydrogel units and assembled into various architectures (e.g., dome, tube, hexagon) (FIG. 31A). Multilayer assembly of microgel layers can be achieved by layer-by-layer stacking (FIG. 31B). Cell viability in the generated units was 97.8% compared with a control group.

As described previously, RAFT is a liquid template self-assembly technology that uses the topography of the gas-liquid interface as a template for assembly of microscale building blocks. The template can be parametrically adjusted by frequencies and accelerations of applied Faraday waves. An experimental platform was designed and implemented that enables generation of one-frequency and two-frequency forced Faraday waves with various wave amplitudes. Stable assembly of microscale building blocks, including mammalian cells, methacrylated gelatin (GelMA) hydrogel units, and polyethylene glycol (PEG) hydrogel units, into various periodic structures (FIG. 31C) was demonstrated. Furthermore, building blocks can be specifically assembled on the different positions of Faraday waves (i.e., antinodes, high-gradient nodes, low-gradient nodes) based on the aspect ratios, density and wettability of the building blocks. These features facilitate assembly of different cell types and microenvironments into specific spatial geometries. Hydrophilic polystyrene beads, PEG and GelMA hydrogel units can be assembled on nodal regions of Faraday waves while polypropylene glycol gel units and copper powders can be assembled on antinodes of Faraday waves. For the same wettability of GelMA hydrogel units, the building blocks with high aspect ratios (e.g., about 3) assembled on high-gradient regions of nodes, while the building blocks with low aspect ratios (e.g., about 0.3) assembled on low-gradient regions of nodes (FIG. 31D). Microscale units were assembled with photo-cross-linking and the entire construct was recovered in a standard cell culture platform. OptiPrep-PBS solution (density, 1.1 g mL⁻¹) containing 5% GelMA and 0.5% (w/v) photoinitiator (Irgacure 2959; CIBA Chemicals) was used as a working solution for generating Faraday waves. The cross-linked hydrogel sheet with the assembled hydrogel units settled to the bottom of the container after 10 s agitation at 40 Hz and 3 g. An additional round of assembly was completed without notable affects from a 450-μm thick hydrogel sheet at the bottom of the liquid chamber (FIG. 25). In addition, theoretical models were developed to describe one-frequency and two-frequency forced Faraday waves (FIG. 31B). Based on the theoretical model, numerical simulations were performed to predict the topography of standing waves. It was found that experimental results were in agreement with numerical simulations.

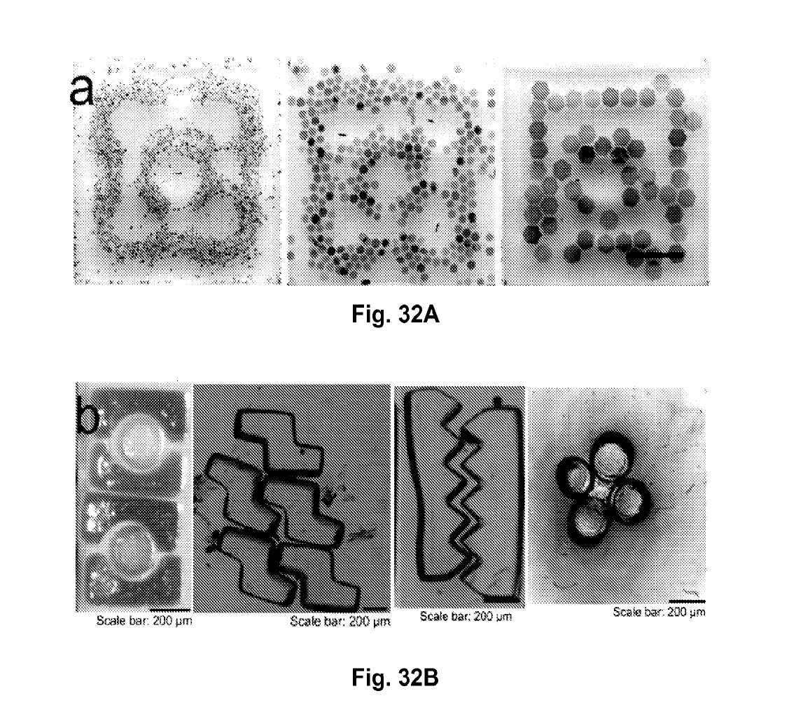

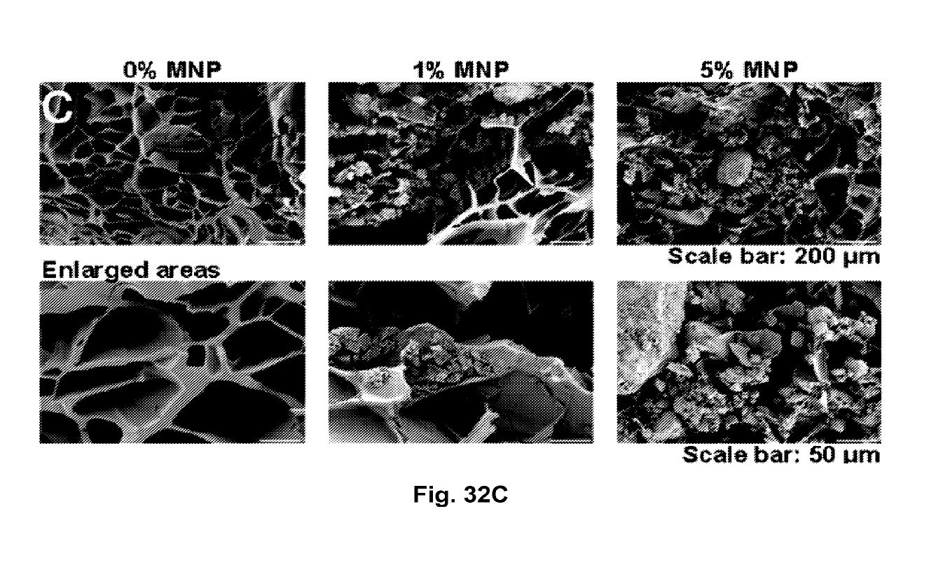

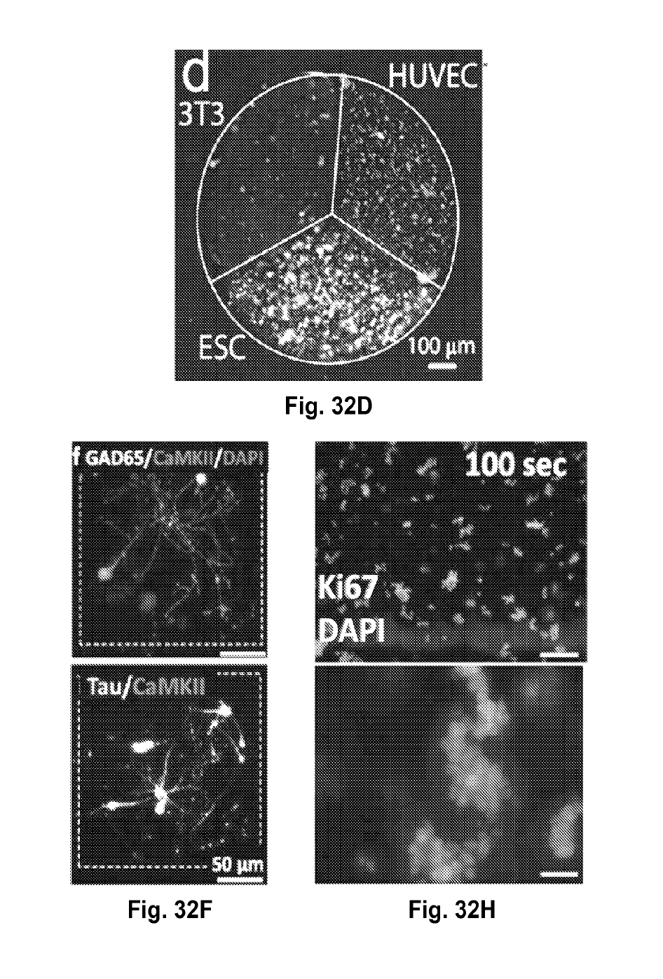

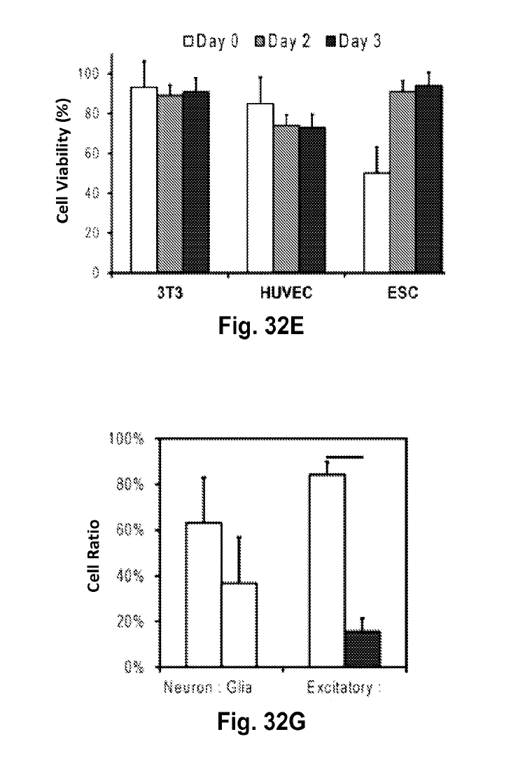

Hydrogels for Tissue Engineering: The ability to engineer hydrogel units with various properties including shapes (e.g., square, hexagon, circle), sizes (ranging from 100 μm to 5 mm), contact angles, porosities, stiffness and magnetic properties was described previously. Geometries of hydrogel units were controlled by the size and the shape of photomasks or PDMS molds for UV photolithography (FIGS. 32A-32B). The porosity, stiffness and density of hydrogel units were controlled by mixing two types of hydrogel with different percentages, or by varying UV exposure dose (i.e., time and intensity). Hydrogel units were functionalized with magnetic properties by loading with magnetic nanoparticles (FIG. 32C) or free radicals.

Encapsulation of different cell types (for example, HUVEC, NIH 3T3 fibroblast cells, and embryonic stem cells) in microscale hydrogel units was accomplished by mixing them into a hydrogel prepolymer solution before molding and subsequent cross-linking (Song, Y. S. et al., Anal. Bioanal. Chem. 395, 185-193; Ling, Y. et al., Lab Chip 7, 756-762). Furthermore, engineering techniques have been developed for microscale, high-precision spatial organization of multiple cell type systems (FIG. 32D). Cell viability in single hydrogel units has been assessed with a fluorescent live/dead assay to investigate the effect of cell concentration on cell viability. Average cell viability was larger than 90% for 3T3 cells and embryonic stem cells, and was larger than 70% for HUVEC (FIG. 32E). Cell proliferation has also been evaluated in PEGDA hydrogel units by Ki67 immunocytochemistry (FIG. 32H).

Digitally specified precision techniques have been developed to co-culture diverse cell types in engineered 3D hydrogel systems. The cellular composition of the cortical brain tissue was recapitulated in terms of neurons and glial cells and the ratio between excitatory neurons to inhibitory neurons was captured within defined in vitro culture. The study quantitatively showed the cell type ratio in hydrogels after 3 weeks of culture. Anti-Tau-1 for neurons (63.16±19.92%), anti-GFAP for glia (36.84±19.92%); anti-CaMKII for excitatory neurons (84.20±10.96%), anti-GAD65 for inhibitory neurons (15.80±10.96%) (FIGS. 32F-32G).The properties of hydrogels can be modified to increase cell adhesion with RGD peptide immobilization, adjustment of the porosity, stiffness and wettability, and through blending different polymeric materials.

Lab-on-a-chip technologies: LOC systems have been developed for virus detection, cell encapsulation, cell capture and controlled cell release. LOC technologies include wide field imaging, lensless imaging, electric sensing, droplet microfluidics and surface modification technologies. For example, a simple and inexpensive microchip ELISA-based detection module was demonstrated that employs a portable detection system, i.e., a cell phone/charge-coupled device (CCD) to quantify an ovarian cancer biomarker, HE4, in urine. Integration of a mobile application with a cell phone enabled immediate processing of microchip ELISA results, which eliminated the need for a bulky, expensive spectrophotometer. The HE4 level detected by a cell phone or a lensless CCD system was significantly elevated in urine samples from cancer patients than healthy controls. Receiver operating characteristic (ROC) analyses showed that the microchip ELISA coupled with a cell phone running an automated analysis mobile application had a sensitivity of 89.5% at a specificity of 90%. Under the same specificity, the microchip ELISA coupled with a CCD had a sensitivity of 84.2%.

A simulated microenvironment was developed for investigating the role of fluidic forces as modulators of metastatic cancer biology using 3D ovarian cancer cells. Changes in the morphological, genetic, and protein profiles of biomarkers associated with aggressive disease were evaluated in the 3D culture environment under controlled and continuous laminar flow. A modulation of biomarker expression and tumor morphology is consistent with increased epithelial-mesenchymal transition that is a critical step in metastatic progression and an indicator of aggressive disease. This observation is originated from hydrodynamic shear stress provided within the designed microfluidic chip. A flow induced, transcriptionally regulated decrease in E-cadherin protein expression and a simultaneous increase in vimentin was also observed, indicating increased metastatic potential. These observations demonstrate that the microfluidic platform developed can provide a flow-informed framework complementary to conventional mechanism-based therapeutic strategies.

General Approach: Embodiments of the RAFT system can address challenges in engineering of vascularized 3D tissue functional units for tissue engineering purpose by merging microscale assembly technologies, LOC technologies, biomaterials sciences and tissue engineering principles. In one embodiment, a Faraday wave-based templated self-assembly technology can be used to construct repeating 3D zonal architectures from microscale hydrogel units. Furthermore, three types of microscale hydrogels units can be used for specific assembly on high-gradient nodes, low-gradient nodes and antinodes of standing waves separately based their densities, wettability and geometries. In another aspect, engineered hydrogel units and the liquid template can be combined to create 3D hydrogel architectures by assembling microscale hydrogels units into a 2D hydrogel sheet and further stacking 2D hydrogel sheets by layer by layer.

In another embodiment, the self-assembly technology can be interfaced with cells for engineering 3D tissue constructs. For example, hepatocytes and liver sinusoidal endothelial cells can be encapsulated into two types of the engineered hydrogel units. 3D hepatic acini can then be derived from cell encapsulating hydrogel units and sacrificial hydrogel units by the microscale self-assembly technology (RAFT) and subsequent tissue culture. Matured engineered 3D tissue constructs can be characterized by investigating distribution of cell types and morphology of the engineered hepatic acini.

In yet another embodiment, LOC technology can be developed for tissue functionalization and functional validation. First, engineered hepatic acini can be interfaced with both oxygenated and deoxygenated cell culture media perfusion systems in an LOC system. Functional validation of the engineered hepatic acini can then be conducted in vitro. Furthermore, functional validation of the engineered hepatic acini can be performed through the use of an animal model.

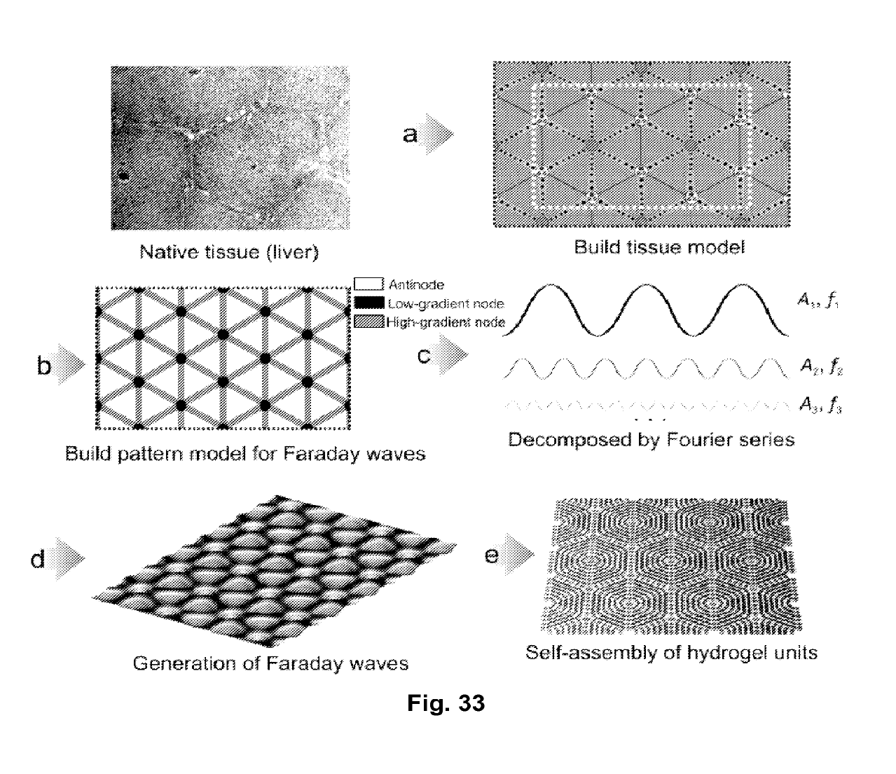

Implementation of a Faraday wave-based templated self-assembly technology for Constructing Repeating Complex 3D Zonal Architectures from Microscale Hydrogel Units: Referring to FIG. 33, 3D hydrogel architecture can be designed based on the target 3D tissue construct. Step a of FIG. 33 illustrates how the target tissue construct can be first simplified into the repeating 3D tissue functional units based on the standard histological model. The tissue functional units can then be decomposed into a set of stacked 2D slices. As illustrated in step b of FIG. 33, each slice can be converted into a corresponding 2D pattern for assembly by correlating different cell types in the tissue functional units to different position on standing waves such as low-gradient nodes, high-gradient nodes and antinodes. As shown in step c of FIG. 33, the 2D patterns can be decomposed into the sum of a series of sine waves by Fourier series. The extracted the frequencies and amplitudes of these sine waves can be used as the frequencies and referential acceleration for generating multiple-frequency forced Faraday waves. One theoretical model can be developed based on 2-D Swift-Hohenberg type equations to predict the topography of the gas-liquid interface for multiple-frequency forced Faraday waves (Lifshitz, R., et al., Physical Review Letters 79, 1261-1264). Another theoretical model was developed based on Gor'kov equations to predict the structure of the self-assembly (Gor'kov, L. P. Sov. Phys. Doklady, 6, 773, 1962). An experimental platform can be built to generate the desired gas-liquid interface based on a theoretical model as illustrated in step d of FIG. 33. Microscale hydrogel units can be used as building blocks for assembly at the gas-liquid interface. To enable specific assembly on standing waves, microscale hydrogels can be engineered in their surface wettability and geometry as illustrated in step e of FIG. 33. Normally, hydrophilic hydrogel units with lower density than the carrier liquid can assemble on the nodal regions of Faraday waves while hydrophobic hydrogel units of a higher density than the carrier liquid can assemble on antinodes of standing waves. Hydrogel units denser than the carrier liquid may not settle down into the liquid during the assembly due to surface tension force. For the same hydrophilic hydrogels, high aspect-ratio hydrogel units can assemble on higher gradient regions of nodes while low aspect-ratio hydrogel units can assemble on lower gradient regions of nodes. To fix the assembled hydrogel units, a small amount of UV cross-linkable hydrogel precursor and photo-initiator can be added to the working solution. The assembled hydrogel units can be cross-linked by a confocal UV exposure system, resulting in formation of a hydrogel layer at the gas-liquid interface. The hydrogel sheet can settled to the bottom of the liquid container by Faraday wave agitation. After the hydrogel sheet settles to the bottom of the chamber, a free gas-liquid interface is available for assembling the next layer of the slice. The 3D hydrogel construct with designed internal architecture can be created by stacking the hydrogel sheets layer by layer.

Platform for the Generation of Desired Topography of Standing Waves as a Liquid Template: An experimental platform that enables one-frequency and two frequency forced Faraday waves has been developed. Based on this design, a new experimental platform can be constructed that enables multiple-frequency forced Faraday waves. This platform can include a vibration generator (e.g., VTS 300, VIBRATION TEST SYSTEMS, AURORA, OHIO) controlled by a program (e.g., LabVIEW). The program can decompose the designed pattern into the sum of sine waves with different frequencies by Fourier series. The extracted frequencies of these sine waves can be used as the frequencies of multiple-frequency forced Faraday waves. In addition, the extracted the amplitudes of these sine waves can be used as the reference for setting accelerations of multiple-frequency forced Faraday waves. The program can also calculate the number of hydrogel units for each hydrogel types based on the designed pattern and the sizes of hydrogel units. An optical system can be built to monitor Faraday waves at the gas-liquid interface based on the refraction (Edwards, W. S., et al., Journal of Fluid Mechanics 278, 123-148). OptiPrep-DPBS solution (density, 1.1 g mL⁻¹) containing 5% GelMA and 0.5% (w/v) photoinitiator (Irgacure 2959; CIBA Chemicals) can be used as the working solution for the experiments.

Engineering of Three Types of Microscale Hydrogels Units for Specific Assembly on High-Gradient Nodes, Low-Gradient Nodes and Antinodes of Standing Waves Separately Based Their Density, Wettability and Geometry: GelMA-PEG hydrogel units can be engineered with a hexagonal shape and a triangle shape for assembly on nodal regions of standing waves, and surface modified GelMA-PEG hydrogel units with a square shape for assembly on antinodes of standing waves. GelMA-PEG hydrogel units can be fabricated by UV photolithography. To fabricate the microscale hydrogel units, the hydrogel prepolymer solution can be prepared by mixing GelMA powder, PEGDMA and 0.5% (w/v) photoinitiator (Irgacure 2959; CIBA Chemicals) in Dulbecco's phosphate buffered saline (DPBS). The ratio of GelMA to PEGDMA can determine the stiffness and degradation rate of ultimate hydrogel units. The prepolymer solution (30 μL) can be pipetted onto a polypropylene slide. Cover glasses (100, 150 or 300 μm thick) can be adhered to the polypropylene slide as spacers to define the thickness of hydrogel units. Another cover glass coated with 3-(trimethoxysilyl)propyl methacrylate (TMSPMA) (Sigma, MO) can be placed on the spacer to spread the prepolymer droplet into a uniform thickness. Photomasks with triangle, square and hexagonal patterns can be used to fabricate hydrogel units with the matched side-length but different aspect ratios. The photomasks can be placed on the TMSPMA cover glass for UV light (360-480 nm) exposure. After the UV polymerization process, the fabricated hydrogel units can be washed with DPBS to remove excess prepolymer residue, stained with 0.5% biocompatible dye for visualization and then washed with DPBS again to remove excess dye. The hydrogel units can be stored in DPBS under ambient conditions prior to use in the experiments. Surface modified GelMA-PEG hydrogel units can be engineered by chemically grafting polypropylene-glycol on GelMA-PEG hydrogel units (Hazer, D. et al, Childs Nerv Syst 28, 839-846; Xia, H., et al., Macromolecular Chemistry and Physics 207, 1945-1952).

The distribution of hydrogel units can be tested on standing waves. A single standing wave can be generated in the center of a liquid chamber as a model to test distribution of hydrogel units. Hydrophilic hydrogel units with different aspect ratio can be tested to achieve complete separation on the standing waves. Finally, one type of hydrophobic hydrogel units and two types of hydrophilic units with complete separation from each other on standing waves can be selected for the experiments described hereinafter.

Creation of 3D Hydrogel Construct by Assembling Microscale Hydrogels Units into a 2D Hydrogel Sheet on the Liquid Template And Further Stacking 2D Hydrogel Sheets with Layer by Layer Assembly: A confocal UV exposure system can be customized to enable cross-link a liquid layer with a high resolution in Z axis. The parameters for settling down the cross-linked hydrogel sheet can be optimized according to the relaxation time for settling and the integrity of the hydrogel sheet by screening vibrational parameters from 40 Hz to 60 Hz in the frequency and 2 g to 8 g in the acceleration.

The liquid carrier chamber with dimensions of 20 mm×20 mm×10 mm can be used in the experiments. The working solution can be first loaded into the chamber. Three types of hydrogel units can be dispersed onto the gas-liquid interface with quantities based on the designed two-dimensional pattern. The vertical vibration with designed frequency can be applied to the liquid-carrier chamber to generate Faraday waves. The acceleration of the vertical vibration can be adjusted to achieve optimal assembly. Once desired assembly is achieved, vertical vibration can be terminated. UV exposure can be performed to cross-link the assembled hydrogel units. The cross-linked hydrogel sheet can be settled down to the bottom of the chamber by vertical vibration. A three-dimensional hydrogel construct can be generated by stacking the hydrogel sheet layer by layer.

Three types of hydrogel units stained with different fluorescence dyes can be successively assembled and stacked. The relative position between hydrogel units in these three layers of hydrogel sheet can be observed by confocal microscope and be compared with designed 3D hydrogel construct to determine the spatial accuracy of the stacking.

In one aspect, the aforementioned system and methods provide a self-assembly platform and related protocols that enables assembly of designed 3D hydrogel construct from microscale hydrogel units. The platform can include software and hardware enable generation of designed Faraday waves, cross-linking assembled hydrogel units by UV exposure, settling down the cross-linked hydrogel sheet by Faraday wave agitation. A commercialized confocal microscope system and multi-photon laser system enables micron resolution and can be used to address technical challenges associated with achieving a desired thickness when cross-linking the assembled hydrogel units at the gas-liquid interface.

In another aspect, an experimental platform is provided for Faraday wave-based templated self-assembly built with microscale hydrogel units as inputs and desired 3D hydrogel architecture as an output.

Interfacing of Self-Assembly Technology with Cells for Engineering 3D Tissue Constructs: The developed technology enables the self-assembly of hydrogel units in predefined motifs on a liquid template. The applicability of this approach in tissue engineering is demonstrated through the liver tissue as a model system. The hepatic acinus is functional repeating units of the liver and is made up of several cells types, where hepatocytes and endothelial cells play most significant roles. Hepatocytes are one of the main cell types of the liver, and they are responsible for a variety of functions such as detoxification, modification, and excretion of exogenous and endogenous substances. Endothelial cells form the capillary network and blood vessels that are supplying oxygenation and exchange of metabolites and nutrients, providing vital support to hepatocytes. In an attempt to mimic the native hepatic acinus, engineered tissue can, in certain embodiments, utilize these two cell types in a well-defined spatial organization.

Hepatocytes can be encapsulated in the hydrogel units that can be assembled on antinodes of standing waves, while liver sinusoid endothelial cells can be encapsulated in the hydrogel units that can be assembled on low-gradient nodal regions of standing waves. Degradation rate of hydrogel units can be optimized by adjusting the ratio of GelMA to PEGDMA to match cellular growth and deposition of newly synthesized ECM. As for the assembly of hydrogel units, cell encapsulating hydrogel units can be loaded into the liquid-carrier chamber with the quantities according to the designed pattern and be assembled on the gas-liquid interface by multiple-frequency forced Faraday waves. The assembled cell-encapsulating hydrogel units can be interconnected by additional cross-linking to form a cell-encapsulating hydrogel sheet. By stacking hydrogel sheets layer by layer, 3D cell-encapsulating hydrogel constructs with designed spatial organization of cell types can be obtained. The assembled cell-encapsulating hydrogel constructs can be further transferred to tissue culture. Proliferated cells can, under certain conditions, completely replace hydrogel scaffold after 2-3 weeks, resulting in formation of engineered 3D tissue construct. Engineered hepatic acinus can be characterized by measuring cell viability, proliferation and distribution as well as biopolymer degradation rate. Spatial organization of two cell types can be compared with the designed architecture.

Engineering of Microscale Cell-Encapsulating Hydrogel Units with Different Cell Types: Both hepatocytes and endothelial cells can be separately encapsulated in defined hydrophobic and hydrophilic hydrogels respectively and cultured in vitro. Cell viability and proliferation assays can be performed to define the optimum hydrogel properties. The synthesis of new extra cellular matrix can be assessed for each hydrogel composition.

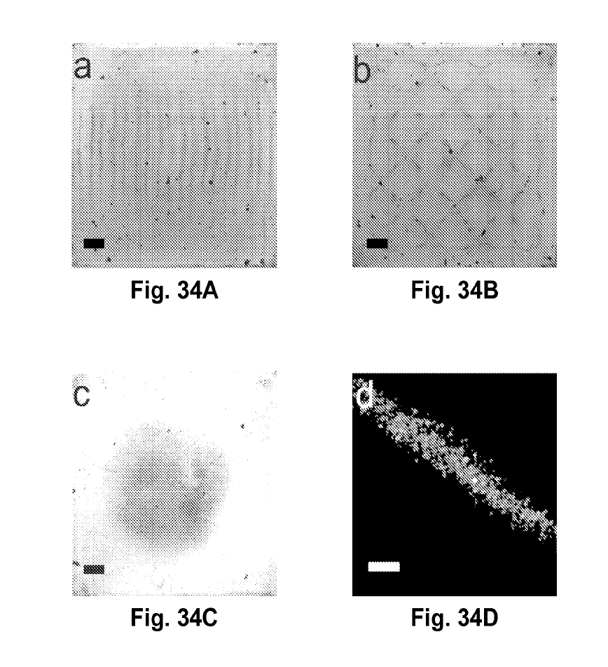

One example of RAFT incorporating cell-encapsulating hydrogel units is shown in FIGS. 34A-34C. Cells were successfully encapsulated in hydrogel units and self-assembled into ordered structures using RAFT. As shown in FIGS. 34A and 34B, the self-assembled structures could be preserved through UV cross-linking. Cell staining with CFSE further confirmed the presence of live cells within the structures (FIG. 34D). Cellular staining with CFSE was accomplished with a CellTrace™ CFSE Cell Proliferation Kit manufactured by Life Technologies™. CFSE passively diffuses into cells and is colorless and nonfluorescent until the acetate groups are cleaved by intracellular esterases to yield highly fluorescent carboxyfluorescein succinimidyl ester. The succinimidyl ester group reacts with intracellular amines, forming fluorescent conjugates that are well retained and can be fixed with aldehyde fixatives.

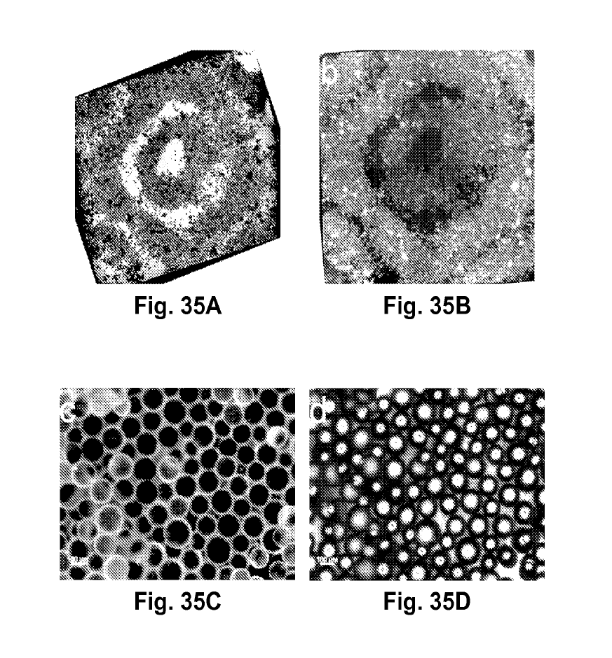

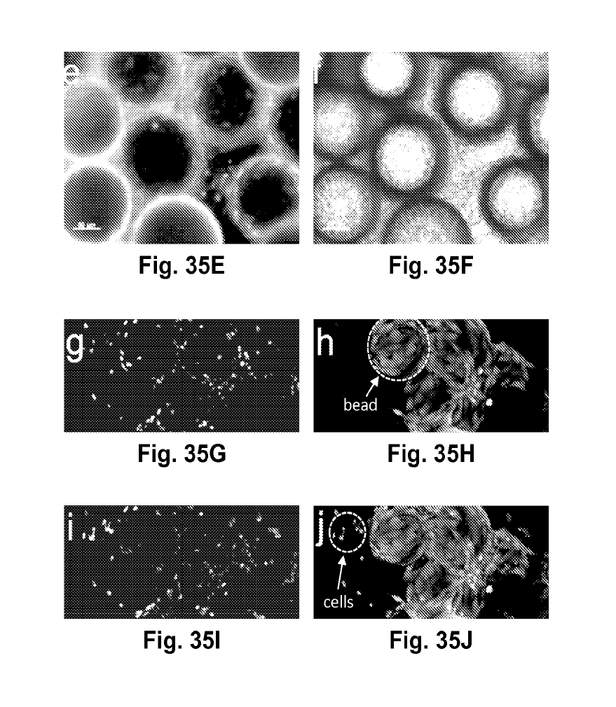

Another example of RAFT incorporating live cells is shown in FIGS. 35A-35J. Live cells were adhered to microcarrier beads that were in turn self-assembled into structure with RAFT as shown in FIGS. 35A and 35B. Fluorescent imaging of cells stained with calcein AM revealed greater than 90% cell viability following assembly and chemical cross-linking FIGS. 35C and 35E). Moreover, staining with either ethidium homodimer-1 (for selective staining of dead cells) or DAPI (for selective staining of nuclei) further illustrated the proliferation of live cells on the assembled and cross-linked microcarrier beads after five days of tissue culture (FIGS. 35G-35J).

Hepatocytes and endothelial cells can be cultured in DMEM and EGM respectively. At a density of 1×10⁷ mL⁻¹, the cells can be suspended in the GelMA-PEG prepolymer solution prepared with different grafting ratios. Cell encapsulating hydrogel units can be fabricated where hepatocytes can be encapsulated in hydrophobic surfaced hydrogel units and endothelial cells can be encapsulated in hydrophilic surfaced hydrogel units. Cell viability of the cell encapsulating hydrogel units can be evaluated at day 0, 1, 3, 5, and 7 with fluorescent live/dead quantification assay. Non-encapsulated cells can be used as control. Proliferation of 3D hydrogel encapsulated cells can be quantified with Alamar blue and MTT assays. After determination of the optimum GelMA-PEG ratio the hydrogel degradation time can be assessed and can be synchronized with synthesis on new ECM. Providing that cell encapsulated 3D hydrogel units are replaced with autologous ECM. Fibronectin, laminin and collagen IV deposition can be monitored as it is the main ECM products of hepatocytes. The synthesis of these ECM components can be quantified over time within the hydrogels with ELISA based assays.

Creation of 3D Hepatic Acini from Cell Encapsulating Hydrogel Units by the Faraday wave-based templated Self-Assembly Technology: As described previously, designed topography of the gas-liquid interface can be generated by multiple-frequency forced Faraday waves. Hydrophobic hydrogel units encapsulating hepatocytes, hydrophilic low-aspect-ratio hydrogel units encapsulating liver sinusoid endothelial cells and hydrophilic high-aspect-ratio hydrogel units can be assembled on antinode, low-gradient nodal regions and high-gradient nodal regions of standing waves respectively. The assembled cell encapsulating hydrogel units can be further cross-linked into a cell-encapsulating hydrogel sheet with a thickness the same as the hydrogel units by a confocal UV exposure system. The cell-encapsulating hydrogel sheet can settle down to the bottom of the liquid carrier chamber by Faraday wave agitation. After the hydrogel sheet settling down to the bottom of the chamber, a free gas-liquid interface is available for next round of assembly and cross-linking. By stacking cell-encapsulating hydrogel sheet layer by layer, 3D cell-encapsulating hydrogel construct with designed cell type distribution can be obtained. By transferring the hydrogel construct to further tissue culture, hydrogel scaffold can be completely replaced cells, resulting in formation of engineered 3D tissue construct with cell distribution like hepatic acini.

A 12-well plate can be used as the liquid carrier chambers for parallel assembly experiments. The 12-well plate can be first loaded with prepolymer solution to a thickness of 10 mm. Two types of cell encapsulating hydrogel units can be loaded into each well in a predetermined quantity. Then, vertical vibration can be applied to the well plate for about 20 seconds with designed frequencies and accelerations for generation of Faraday waves and assembly of hydrogel units. A confocal microscope system equipped with a UV exposure system can be used to selectively cross-link the assembled cell encapsulating hydrogel units at the gas-liquid interface with the same thickness of hydrogel units. The cell encapsulating sheet can settle down to the bottom of the wells by Faraday wave agitation. The above process can be repeated a number of times (e.g., 5 times), resulting in a hydrogel block with a final thickness (e.g., 0.5 mm). The prepolymer solution can be removed. The wells can be washed with PBS (e.g., 5 times) and then replenished with cell culture media. Cell encapsulating 3D hydrogel block can be cultured on a transwell system to ensure culture media exchange from both top and bottom sides of the 3D hydrogel block. Tissue culture can be conducted until the hydrogel scaffolds are completely replaced by the cells.

Structural Characterization of the Engineered 3D Tissue Constructs: Hydrogels encapsulated with hepatocytes and HUVECs can be assembled and further maturated in vitro for about 2-3 weeks, or until the replacement of hydrogels with autologous ECM takes place. The engineered tissue construct can be analyzed in cell proliferation, cell viability, cellular distribution and morphology to validate the compliances with the native tissues. Cell proliferation, growth and viability can be assed with Ki67, Alamar blue, MTT and live/dead staining. The morphology of generated tissue units can be characterized with histological staining for CD31, VE-Cadherin, SEM and TEM microscopy.

Cell necrosis at the core of stacked sheets can occur due to limited oxygen and nutrient/waste transfer. In such case, the thickness of each of the hydrogel units can be decreased to improve the exchange of oxygen and nutrient/waste. It is anticipated that cell proliferation assays and cell viability assays can prove cells in the engineered tissue construct are in a good growth condition. It is further anticipated that histological staining and SEM/TEM can be used to confirm that the engineered 3D hepatic acini have the same cell distribution and morphology with the designed architecture.

In one aspect, the system provides that the spatial distribution of cells in the engineered 3D tissue construct is similar to the designed architecture.

Lab-On-A-Chip Technology for Tissue Functionalization and Functional Validation: Heamodynamic stimulation of tissue engineered hepatic graft can be beneficial for achieving native like tissue functional units. Functional vascularization can also be beneficial for engraftment and support of the hepatocytes. Engineered hepatic acini can be interfaced with mimicking microphysiological environment in a LOC platform. Oxygenated and deoxygenated cell culture media can be perfused at the physiological flow rate through hepatic acini to mimic native hepatic acini.

Basic liver functionality can be evaluated through analysis of urea production, albumin and steroid hormones secretion. Beside these, glucogenesis, cholesterol synthesis, glycolysis, lipogenesis and cytochrome P-450 based drug detoxification are also among the functions of liver tissue units. Functionality and morphology of generated vascularized tissue units can be validated by assessing named functionalities with immunocytochemistry, ELISA and molecular biology based assays.

Interfacing of Engineered Hepatic Acini with Both Oxygenated and Deoxygenated Cell Culture Media Perfusion System in a Lab-On-A-Chip Platform: A PDMS chip can be fabricated to accommodate hepatic acini according to the standard protocols. A commercialized perfusion system (e.g., RCMWTM, Synthecon, Houston, Tex.) can be interfaced with engineered hepatic acini via ultra-fine needle (e.g., OD, 60 μm; ID, 20 μm) and capillary tubes (e.g., OD, 80 μm; ID, 40 μm). Cell culture media with and without oxygen can perfuse through the engineered hepatic acini for mimicking hepatic arteries and central veins. The perfused solution can be collected for further metabolomics analysis.

In one aspect, the engineered hepatic acini can be interfaced with a microfluidic perfusion system via capillary tubes. Oxygenated and deoxygenated cell culture media can be perfused through the engineered hepatic acini just like the native hepatic acini. The solutions that are perfused through the hepatic acini can be used for further analysis. In another aspect, native hepatic acini from porcine liver can be used as an alternative cell type.

Functional Validation of the Engineered Hepatic Acini: Examples of functional assays for hepatic acini include urea production, albumin and steroid hormones secretion. Drug metabolism of the engineered tissue can be analyzed by measuring the distribution of cytochrome P-450 enzyme activity and compared with native hepatic acini.

Cell specific characterization can be performed with immunocytochemistry by staining for CD31 (PECAM) and VE-Cadherin for endothelial cells and anti-hepatocyte E-Cadherin and anti-albumin for hepatocytes. Engineered tissue units can be also analyzed for functionality by validating albumin secretion with ELISA, the drug metabolism can be quantitatively assessed via CYP450-1A1 enzyme activity and urea assays.

In one aspect, alcohol can be metabolized in the hepatic acini by the enzyme cytochrome P450IIE1 (CYP2E1). The alcohol concentration in the collected well plate can be lower than that the input. In addition, distribution of P450IIE1 can be visualized by fluorescence antibody staining. In another aspect the distribution of P450IIE1 in Zone III can be higher than Zone II in hepatic acini, as for native hepatic acini.

In vivo Functional Validation of the Engineered Hepatic Acini: Heamodynamic stimulation of tissue engineered hepatic graft can be beneficial for achieving native like tissue functional units. Functional vascularization can also be beneficial for engraftment and supporting the hepatocytes. Performance of in vitro engineered tissue functional units can be further tested in vivo whether it is completely functional liver tissue. To observe the true potential of encapsulated hepatocytes and pre-vascularization formed by endothelial cells, generated hepatic acini can be subcutaneously implanted in immune-compromised nude mouse model.

After in vitro assembly, the maturated hepatic acini can be subcutaneously implanted in an immune-compromised nude mouse to avoid any immune reaction rising from host to human cells. Implanted graft can be analyzed for anastomosis of pre-vascularization and host vasculature. To achieve this, serial implantations can be performed and the kinetics observed at 1, 2, 4, 6 and 24 hours post implantation. To visualize the perfused vasculature fluorescein-conjugated dextran can be infused in combination with CD31 antibody. Anastomosis can be confirmed with whole-mount immune staining of explants. The evaluation of liver functions tissue engineered liver functional units can be implanted up to 8 weeks. Histological analyses of explanted grafts can be performed for identification of present human cells with in situ hybridization of Alu gene sequences. Functionality of implanted graft can be assessed with analysis of human albumin and human serum alpha-1-antitrypsin. Drug metabolism activity can be evaluated in conditions where ketoprofen and debrisoquine were administrated to mice. Ketoprofen and debrisquine are metabolized in different manner in humans and mice. Human specific metabolites can be detected from serum and urea samples of implanted mice.

In one aspect, engineered hepatic acini are validated in their functions at the tissue/organ level.

### EXAMPLE 4

### Further Application of RAFT to Tissue Engineering

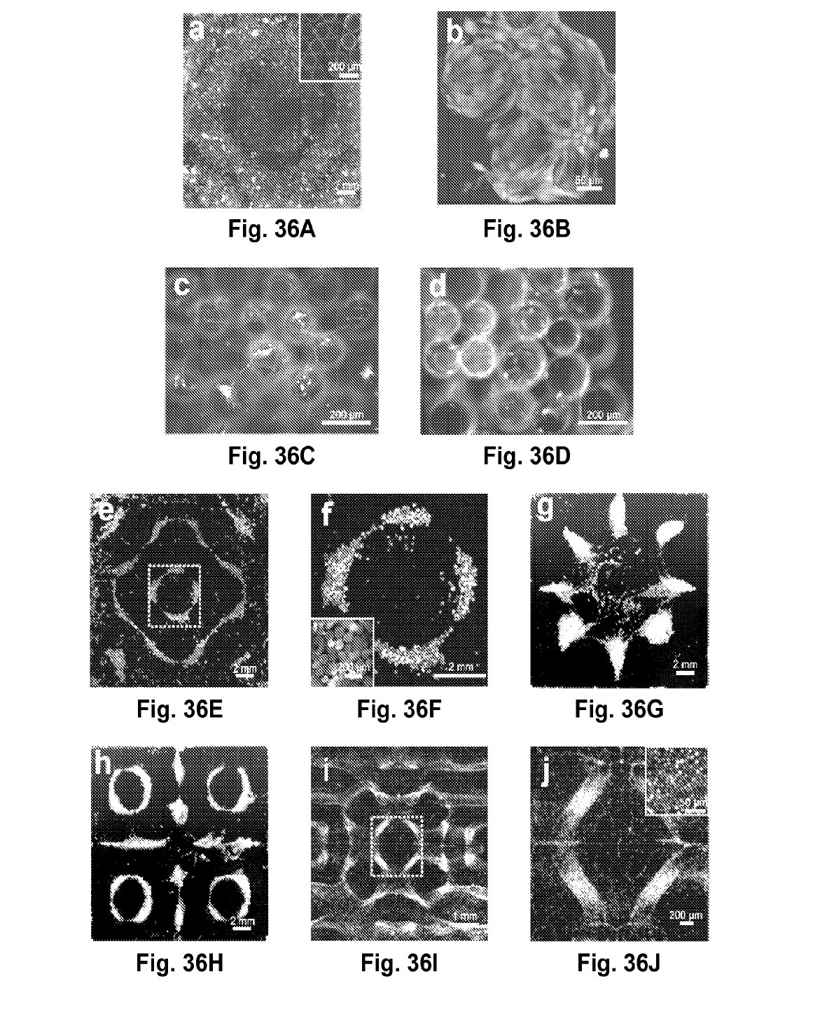

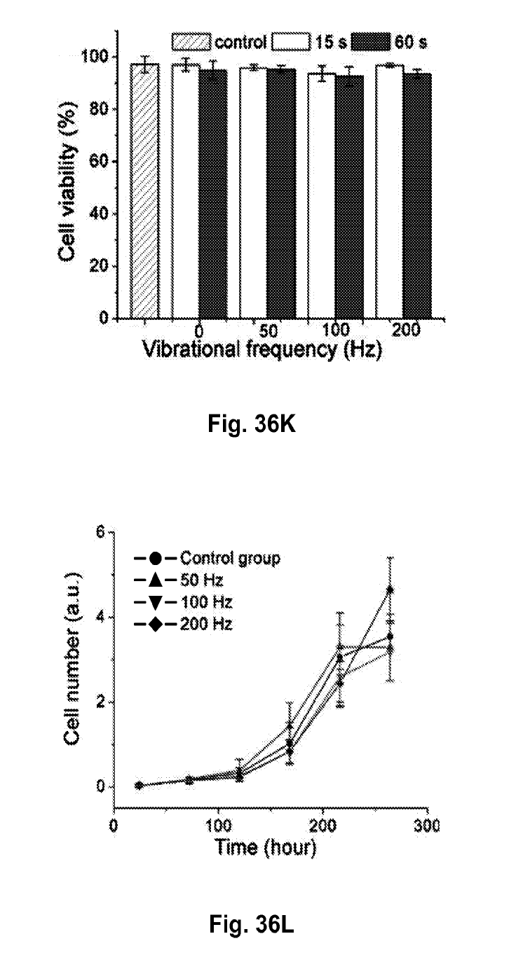

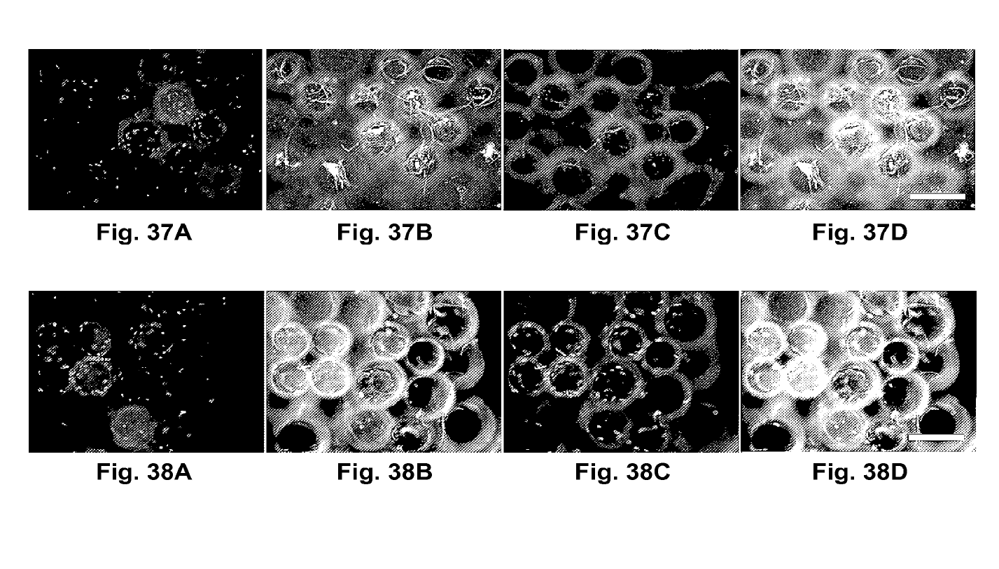

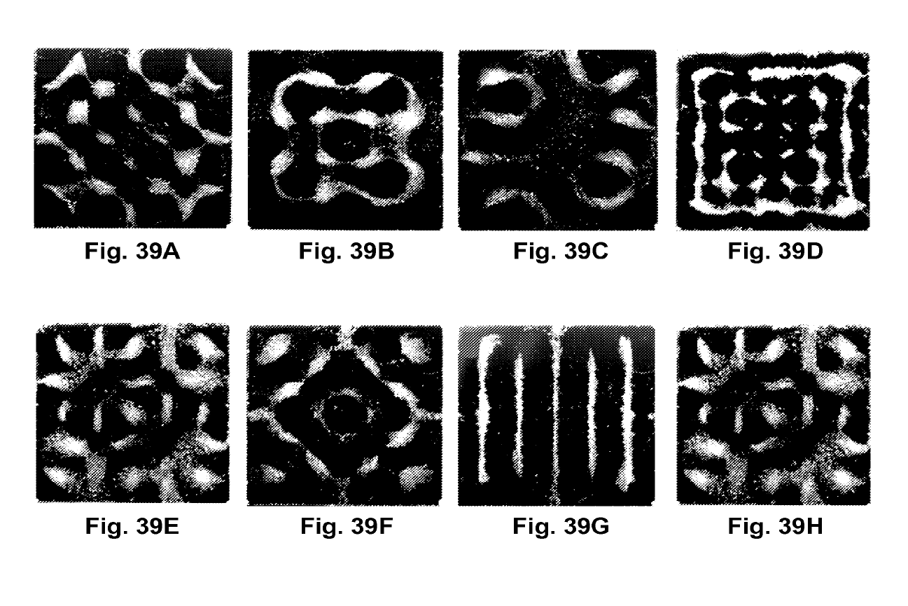

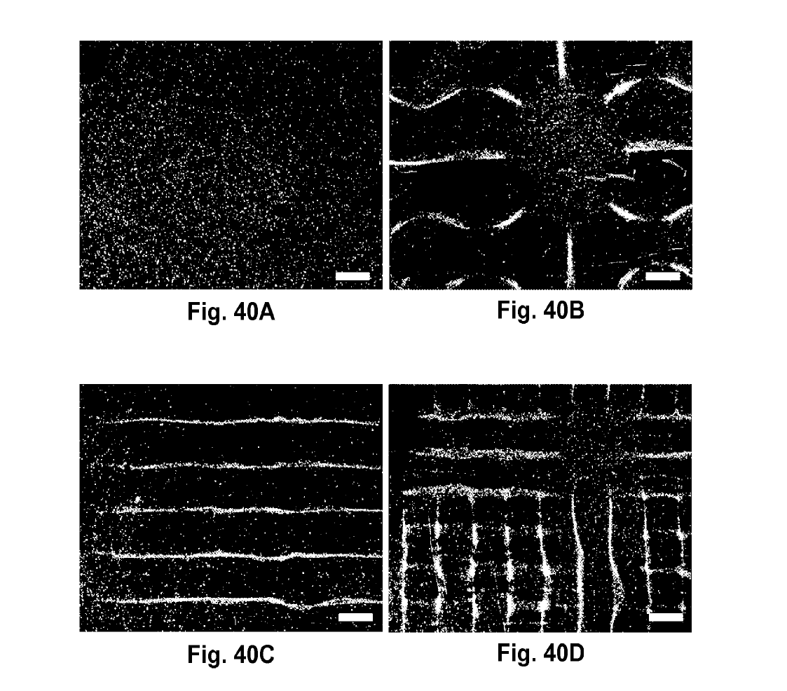

Yet another example of RAFT incorporating live cells is shown in FIGS. 36A-36L. Polystyrene beads were used as microcarriers for cell assembly as shown in FIG. 36A. Cell-seeded microcarrier beads were assembled into various patterns and the formed motif of the cell-seeded beads was immobilized by chemical cross-linking with fibrinogen and thrombin. High cell viability was confirmed with live/dead assays after three-days of culture as shown in FIG. 36B. In one aspect, assembly of neuron-seeded microcarrier beads into large-scale 3D neural structures may contribute to the development of in vitro models for understanding the wiring and mapping of neurons. Neuron-seeded beads were assembled for the generation of 3D neural structures that tested positive for markers such as Nestin, NeuN and MAP-2 (FIGS. 36C-36D, 37A-37D and 38A-38D). Patterning cell spheroids into various shapes may be of significance for tissue engineering due to the capability of the fusion of spheroids into micro-tissues. In one aspect, RAFT was used to simultaneously assemble ˜10³ cell spheroids (FIGS. 36E-36H and 39A-39H). RAFT was further applied for the simultaneous assembly of ˜10⁶ cells into various patterns by (FIGS. 36I-36J and 40A-40D). Cytocompatibility of RAFT was determined with live/dead assays for cell viability and Alamar-Blue assays for cell proliferation. Within the first 24 hours, cells exposed to 15 and 60 second agitations at 50, 100 and 200 Hz did not show significant differences in viability as compared with a control group (FIG. 36K). Observations after 11 days of cell culture further indicated that the cells exposed to standing waves exhibited no significant differences in proliferation as compared with the control group (FIG. 36L).

For generation of cell spheroids, NIH 3T3 mouse fibroblast cells were cultured as described herein, harvested and plated in 60 mm non-adherent Petri dishes with 1.6 million cells per dish. Cell spheroids with an average size around 200 μm were observed after 2 days of culture. For the formation of neuron-seeded microcarrier beads, primary neuronal cells were isolated from neural cortex of postnatal 1 day old (PD 1) Sprague Dawley® rats acquired from Charles River Labs, USA in accordance with institutional guidelines for care and use of animals. Cortex tissue was dissociated by trituration after digestion using papain (20 U ml⁻¹). Cells were then suspended in DMEM/F12 media supplemented with Glutamax, N2 and B27 (Life Technologies), bFGF and EGF.

Synthemax II microcarrier beads from Fisher Scientific Inc. were coated with 1 μM laminin at 37° C. over night. Harvested primary cortical neurons were seeded on laminin coated beads with a seeding number of around 1.5 million cells per well in a non-adhering 6-well plates for 5 days. Neuron-seeded microcarrier beads were harvested and suspended in fresh DMEM/F12 media with a final concentration of 80% (v/v) before use. Neuron-seeded microcarrier beads were assembled and further stabilized by chemical cross-linking. The stabilized neuron-seeded microcarrier beads were cultured in neural culture media in the cell incubator at 37° C. for 14 days before final characterization.

NIH 3T3 fibroblast cells were stained with CellTrace™ CFSE for fluorescence microscopic imaging in the assembly. CFSE staining solution was prepared in pre-warmed PBS with a final concentration of 25 μM. Cells were harvested according to standard protocol and incubated in CFSE staining solution at 37° C. for 20 min. The cells were further incubated with pre-warmed fresh culture medium at 37° C. for another 30 min. The cells were washed with PBS for three times and suspended in the culture medium with a final concentration of 10 million mL⁻¹ before use.

Fibrin hydrogels containing neurons were fixed with 4% paraformaldehyde for 20 min at room temperature and washed with excessive PBS. Hydrogels were permeabilized with 0.3% Triton-X 100 and blocked with 1% BSA. Cells were stained overnight at 4° C. for primary antibodies: anti-NeuN, anti-Nestin, anti-MAP2. Samples were washed and stained 2 h at room temperature for secondary antibodies goat anti-mouse Alexa Fluor® 488 and goat anti-rabbit Alexa Fluor® 568 antibodies, DAPI was used as nuclear counter staining. Cell viability and proliferation assays: Cell viability test was performed with LIVE/DEAD® viability/cytotoxicity kit from Molecular Probes. The LIVE/DEAD® staining solution was prepared by mixing 20 μL ethidium bromide and 5 μL calcein in the in 10 mL PBS.

NIH 3T3 fibroblast cells were harvested and prepared with final cell density as 10,000,000 cells mL⁻¹ in DMEM cell culture medium. OptiPrep-PBS (1.2 g mL⁻¹) was prepared as working solution for assembly. Cell suspension was added to the working solution and agitated as described previously. Treated cells were sampled and seeded into 96 well plates with a seeding number of ˜9,800 cells per well. After 24 hours of cell culture, the cells in each well were incubated with 40 μL staining solution in the 37° C. incubator for 20 min. After the incubation, the staining solution was diluted with 200 μL PBS. Cell proliferation tests were performed with Alamar Blue and was measured in a microplate reader. Fluorescence imaging was performed with an inverted microscope or confocal microscope.

Both chemical cross-linking and photo-cross-linking can be used to stabilize assembled structures. For the chemical cross-linking, OptiPrep™-PBS solution (density, 1.1 g mL⁻¹) with human fibrinogen (10 mg ml⁻¹) was prepared as a working solution for assembly. The cross-linking was performed by adding thrombin (10 μL, 12.5 IU ml⁻¹, Sigma). For the photo cross-linking, GelMA hydrogel prepolymer and photoinitiator (Igracure™ 2959; CIBA Chemicals) were dissolved in the OptiPrep™-PBS solution with final concentrations of 2.5% (w/v) and 0.5% (w/v) respectively. The cross-linking was performed by photo exposure (wavelength, 360-380 nm; power 700 mW cm⁻²; exposure time, 90 s). Images and data were analyzed and plotted using image analysis software, and numerical simulation, analysis and modeling software.

Each reference identified in the present application is herein incorporated by reference in its entirety.

While present inventive concepts have been described with reference to particular embodiments, those of ordinary skill in the art will appreciate that various substitutions and/or other alterations may be made to the embodiments without departing from the spirit of present inventive concepts.

Accordingly, the foregoing description is meant to be exemplary, and does not limit the scope of present inventive concepts.

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

What is claimed is:

1. A method of manufacturing a structure, comprising:
providing a chamber containing a gas-liquid interface or liquid-liquid interface;
dispersing a plurality of floaters at the gas-liquid interface or liquid-liquid interface;
oscillating the chamber along an axis orthogonal to the gas-liquid interface or liquid-liquid interface, thereby generating a standing wave at the gas-liquid interface or liquid-liquid interface;
allowing the floaters to self-assemble; and
linking the floaters,
wherein the standing wave is formed by a parametric instability on the surface of the liquid.

2. The method of claim 1, wherein the standing wave is a Faraday wave.

3. The method of claim 1, wherein the floaters have a diameter of about 0.1 μm to about 1 m.

4. The method of claim 1, wherein the floaters have a diameter of about 10 μm to about 5 mm.

5. The method of claim 1, wherein the floaters are at least one of a biological sample, a chemical sample and a non-biomaterial unit,
wherein the biological sample is at least one of microorganisms, cells, cell clusters, cell spheroids, cell fragments, viruses, bacteria, fungi, peptides, nucleic acids, proteins, carbohydrates, secreted cellular products and exosomes,
wherein the chemical sample is at least one of biomaterial units, hydrogel units and polymer units, and
wherein the non-biomaterial unit is at least one of semiconductor units and metallic units.

6. The method of claim 5, wherein at least a portion of the floaters encapsulate or are coated with the biological sample.

7. The method of claim 6, wherein the biological sample is a microorganism, a cell, a cell cluster, a cell spheroid, a cell fragment, a virus, a bacteria, a fungi, a peptide, a nucleic acid, a protein, a carbohydrate, a secreted cellular product, or an exosome.

8. The method of claim 1, wherein the step of linking the plurality of floaters further comprises photo cross-linking , UV cross-linking, chemical cross-linking , thermo cross-linking, surface molecule recognition-based linking, or geometric shape-based linking.

9. The method of claim 8, further comprising:
forming a monolayer structure following the step of linking the plurality of floaters;
repeating the method of claim 8 to produce a plurality of monolayer structures; and
stacking the monolayer structures layer by layer into a 3D architecture.

10. The method of claim 9, further comprising:
culturing the 3D architecture, thereby forming 3D tissue constructs.

11. The method of claim 1, further comprising:
forming a monolayer structure following the step of linking the plurality of floaters.

12. A structure made by the method of claim 1.

13. A system for manufacturing a structure, comprising:
a chamber having a bottom surface;
a liquid disposed in the chamber;
a plurality of floaters disposed on the liquid;
an oscillating mechanism configured to oscillate the chamber along an axis orthogonal to the gas-liquid interface or liquid-liquid interface, thereby generating a standing wave at the gas-liquid interface or liquid-liquid interface; and
a linking mechanism configured to link the plurality of floaters;
wherein the standing wave is formed by a parametric instability on the surface of the liquid.

14. The system of claim 13, wherein the standing wave is a Faraday wave.

15. The system of claim 13, wherein the plurality of floaters have a diameter of about 10 μm to about 5 mm.

16. The system of claim 13, wherein the floaters are at least one of a biological sample, a chemical sample and a non-biomaterial unit,
wherein the biological sample is at least one of microorganisms, cells, cell clusters, cell spheroids, cell fragments, viruses, bacteria, fungi, peptides, nucleic acids, proteins, carbohydrates, secreted cellular products and exosomes,
wherein the chemical sample is at least one of biomaterial units, hydrogel units and polymer units, and
wherein the non-biomaterial unit is at least one of semiconductor units and metallic units.

17. The system of claim 16, wherein at least a portion of the floaters encapsulate the biological sample.

18. The system of claim 17, wherein the biological sample is a microorganism, a cell, a cell cluster, a cell spheroid, a cell fragment, a virus, a bacteria, a fungi, a peptide, a nucleic acid, a protein, a carbohydrate, a secreted cellular product, or an exosome.

19. The system of claim 13, further comprising:
a substrate for stacking a plurality of monolayer structures layer by layer into a 3D architecture.

20. The system of claim 19, further comprising:
a culture chamber for culturing the 3D architecture into 3D tissue constructs.

21. A method of manufacturing a structure, comprising:
providing a chamber containing a gas-liquid interface or liquid-liquid interface;
dispersing a plurality of floaters at the gas-liquid interface or liquid-liquid interface, the floaters having a diameter of about 10 μm to about 5 mm;
oscillating the chamber along an axis orthogonal to the gas-liquid interface or liquid-liquid interface, thereby generating a Faraday wave at the gas-liquid interface or liquid-liquid interface, the Faraday wave formed by a parametric instability on the surface of the liquid;
allowing the floaters to self-assemble; and
linking the floaters to form the structure.
